(19) United States
(12) Patent Application Publication
(10) Pub. No.: US 2026/0108205 A1
(43) Pub. Date: Apr. 23, 2026

(54) WEARABLE DEVICE, METHOD, AND NON-TRANSITORY COMPUTER READABLE STORAGE MEDIUM TO PROVIDE NOTIFICATION BASED ON DISTANCE BETWEEN WEARABLE DEVICE AND EXTERNAL ELECTRONIC DEVICE

(71) Applicant: SAMSUNG ELECTRONICS CO., LTD., Suwon-si (KR)

(72) Inventors: Eunsun KIM, Suwon-si (KR); Taehan LEE, Suwon-si (KR)

(21) Appl. No.: 19/288,139

(22) Filed: Aug. 1, 2025

Related U.S. Application Data
(63) Continuation of application No. PCT/KR2025/010377, filed on Jul. 15, 2025.

(30) Foreign Application Priority Data
Oct. 23, 2024 (KR) 10-2024-0146084
Dec. 4, 2024 (KR) 10-2024-0178970

(51) Int. Cl. A61B 5/00

(52) U.S. Cl. CPC A61B 5/4561; A61B 5/681; A61B 5/7246; A61B 5/7405; A61B 5/742; A61B 5/7455; A61B 5/746; A61B 2560/0214; A61B 2560/0238; A61B 2562/0219; A61B 2562/0257

(57) ABSTRACT

A wearable device includes memory storing instructions, a display, communication circuitry, and at least one processor. The instructions cause the wearable device to, while being connected to an external electronic device through the communication circuitry, obtain information associated with a distance between the wearable device and the external electronic device, by using the information, identify whether the distance is included in a range that is set according to a reference distance between the wearable device and the external electronic device, based on obtaining the distance outside the range identify time during which the distance has been maintained outside the range, and based on identifying the time greater than threshold time, display, through the display, a first user interface (UI) object indicating that a posture of a user of the wearable device is corresponding to a first posture, and such that the reference distance is obtained before the information is obtained.

### CROSS-REFERENCE TO RELATED APPLICATIONS

This application is a continuation of International Application No. PCT/KR2025/010377 designating the United States, filed on Jul. 15, 2025, in the Korean Intellectual Property Receiving Office and claiming priority to Korean Patent Application Nos. 10-2024-0146084, filed on Oct. 23, 2024, and 10-2024-0178970, filed on Dec. 4, 2024, in the Korean Intellectual Property Office, the disclosures of each of which are incorporated by reference herein in their entireties.

### BACKGROUND

The present disclosure relates to a wearable device, a method, and a non-transitory computer readable storage medium to provide a notification based on a distance between a wearable device and an external electronic device.

A wearable device may include a sensor. The wearable device may measure a distance between an external electronic device and the wearable device through the sensor. The wearable device may measure the distance through a time of flight (ToF) technique. The wearable device may measure the distance between the external electronic device and the wearable device by using a received signal strength indicator (RSSI). The wearable device may measure the distance according to the RSSI using communication circuitry.

The above-described information may be provided as a related art for the purpose of helping understanding of the present disclosure.

No argument or decision is made as to whether any of the above description may be applied as a prior art related to the present disclosure.

### SUMMARY

A wearable device is described. The wearable device may comprise memory comprising one or more storage media storing instructions. The wearable device may comprise a display. The wearable device may comprise communication circuitry. The wearable device may comprise at least one processor comprising processing circuitry. The instructions, when executed by the at least one processor individually or collectively, may cause the wearable device to, while being connected to an external electronic device through the communication circuitry, obtain information associated with a distance between the wearable device and the external electronic device. The instructions, when executed by the at least one processor individually or collectively, may cause the wearable device to, by using the information, identify whether the distance is included in a range that is set according to a reference distance between the wearable device and the external electronic device. The reference distance may be obtained before the information is obtained. The instructions, when executed by the at least one processor individually or collectively, may cause the wearable device to, based on obtaining the distance outside the range, identify time during which the distance has been maintained outside the range. The instructions, when executed by the at least one processor individually or collectively, may cause the wearable device to, based on obtaining the distance outside the range, based on identifying the time greater than threshold time, display, through the display, a first user interface (UI) object indicating that a posture of a user of the wearable device is corresponding to a first posture.

A method is provided. The method may be executed in a wearable device with a camera and communication circuitry. The method may comprise, while being connected to an external electronic device through the communication circuitry, obtaining information associated with a distance between the wearable device and the external electronic device. The method may comprise, by using the information, identifying whether the distance is included in a range that is set according to a reference distance between the wearable device and the external electronic device. The reference distance may be obtained before the information is obtained. The method may comprise, based on obtaining the distance outside the range, identifying time during which the distance has been maintained outside the range. The method may comprise, based on obtaining the distance outside the range, based on identifying the time greater than threshold time, displaying, through the display, a first user interface (UI) object indicating that a posture of a user of the wearable device is corresponding to a first posture.

A non-transitory computer readable storage medium is provided. The non-transitory computer readable storage medium may store one or more programs. The one or more programs may comprise instructions to, when executed by a wearable device with a display and communication circuitry, cause the wearable device to, while being connected to an external electronic device through the communication circuitry, obtain information associated with a distance between the wearable device and the external electronic device. The one or more programs may comprise instructions to, when executed by the wearable device, cause the wearable device to, by using the information, identify whether the distance is included in a range that is set according to a reference distance between the wearable device and the external electronic device. The reference distance may be obtained before the information is obtained. The one or more programs may comprise instructions to, when executed by the wearable device, cause the wearable device to, based on obtaining the distance outside the range, identify time during which the distance has been maintained outside the range. The one or more programs may comprise instructions to, when executed by the wearable device, cause the wearable device to, based on obtaining the distance outside the range, based on identifying the time greater than threshold time, display, through the display, a first user interface (UI) object indicating that a posture of a user of the wearable device is corresponding to a first posture.

### BRIEF DESCRIPTION OF THE DRAWINGS

FIG. 1 illustrates an example of an environment including a wearable device.

FIG. 2 is a simplified block diagram of an exemplary wearable device.

FIG. 3 is a flowchart illustrating an operation of a wearable device that provides a notification based on a distance between the wearable device and an external electronic device.

FIG. 4 illustrates an exemplary operation of a wearable device identifying a tilt of an external electronic device and a position of the wearable device.

FIG. 5 illustrates an exemplary operation of a wearable device for obtaining a reference distance between the wearable device and an external electronic device.

FIG. 6 illustrates an example of operations executed in a wearable device and an external electronic device.

FIGS. 7A and 7B illustrate an exemplary operation of a wearable device displaying a screen for a setting for providing a notification.

FIGS. 8A and 8B illustrate an exemplary operation of a wearable device displaying a screen associated with a notification.

FIGS. 9A to 9C illustrate an exemplary operation of a wearable device displaying a UI object.

FIG. 10 is a block diagram of an electronic device in a network environment according to various embodiments.

FIGS. 11A and 11B illustrate perspective views of an exemplary electronic device according to an embodiment.

FIG. 12 illustrates an exploded perspective view of an exemplary electronic device according to an embodiment.

### DETAILED DESCRIPTION

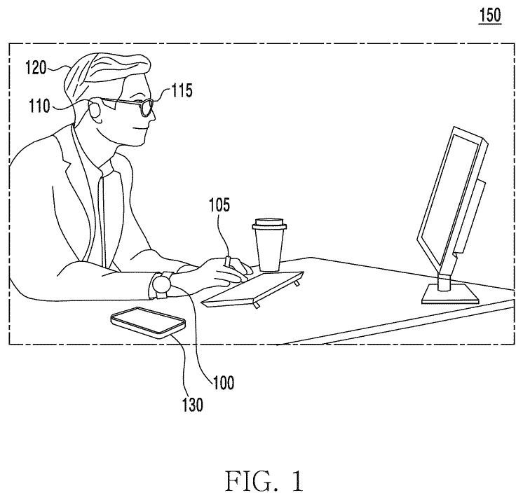

FIG. 1 illustrates an example of an environment including a wearable device.

Referring to FIG. 1, an environment 150 may include a wearable device 100, external electronic devices (e.g., an external electronic device 115, an external electronic device 105, and an external electronic device 130), and a user 120. For example, the wearable device 100 may be worn by the user 120. For example, the wearable device 100 may be worn on a hand or a wrist of the user 120. For example, the external electronic device 110 may be worn on a head or an ear of the user 120. For example, the wearable device 100 may include a smart watch. For example, the wearable device 100 may include a smart ring. For example, the user 120 may wear the external electronic device 110, the external electronic device 115, the external electronic device 105, and the wearable device 100. For example, the external electronic device 110 may be an earphone. For example, the external electronic device 115 may include an augmented reality (AR) glass. For example, the external electronic device 115 may include a video see-through (VST) device. For example, the external electronic device 105 may be a smart ring. For example, the external electronic device 130 may be a smartphone.

For example, the user 120 may perform a task in a state of wearing a plurality of wearable devices including the wearable device 100. For example, the user 120 may read a book or edit a document using a personal computer (PC) in a state of wearing the plurality of wearable devices. For example, the user 120 may change a posture while performing the task as time passes. For example, the user 120 may change the posture while performing the task from a correct posture to an incorrect posture. For example, the user 120 may change the posture while performing the task from the incorrect posture to the correct posture. Correct posture involves aligning the body to minimize stress on muscles and joints. When standing, this means the head is balanced over one's shoulders, the shoulders are relaxed and down, and one's spine maintains its natural curves. When sitting, correct posture involves having the back supported, feet flat on the floor, and knees at a right angle. When sitting, correct posture involves sitting with one's back straight against the back of the chair, maintaining the natural curves of the spine. Incorrect posture or bad posture may refer to a body position where the spine and limbs are misaligned, placing extra strain on muscles and joints. Incorrect posture may be characterized by positions like rounded shoulders, forward head posture, or a slouched spine.

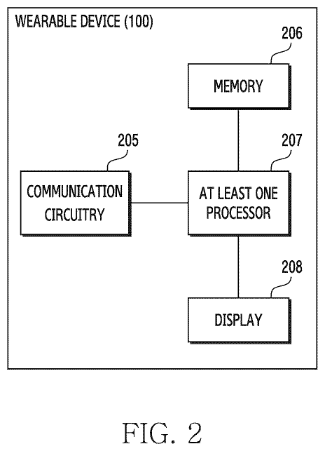

The wearable device 100 may obtain information on the posture of the user 120 while being worn by the user 120. For example, the wearable device 100 may measure a distance between an external electronic device and the wearable device 100 using a sensor included in the wearable device 100. For example, the wearable device 100 may measure the distance between an external electronic device (e.g., the external electronic device 110) and the wearable device 100 using a time of flight (ToF) sensor. For example, the wearable device 100 may measure the distance using light reflected from the external electronic device 110. For example, the wearable device 100 may measure the distance by identifying a difference in a phase of the light through the ToF sensor. For example, the wearable device 100 may measure the distance by using a difference in time when the light reaches the ToF sensor. However, it is not limited thereto. For example, the wearable device 100 may measure the distance between the wearable device 100 and the external electronic device 110 using a received signal strength indicator (RSSI). For example, the wearable device 100 may identify the distance between the wearable device 100 and the external electronic device 110 by measuring an intensity of a signal transmitted from the external electronic device 110. For example, based on the signal received from the external electronic device 110, the wearable device 100 may identify the distance by identifying an amount of a reduced intensity of a signal transmitted to the external electronic device 110. For example, the wearable device 100 may identify the distance through the RSSI using communication circuitry (e.g., communication circuitry 205 of FIG. 2).

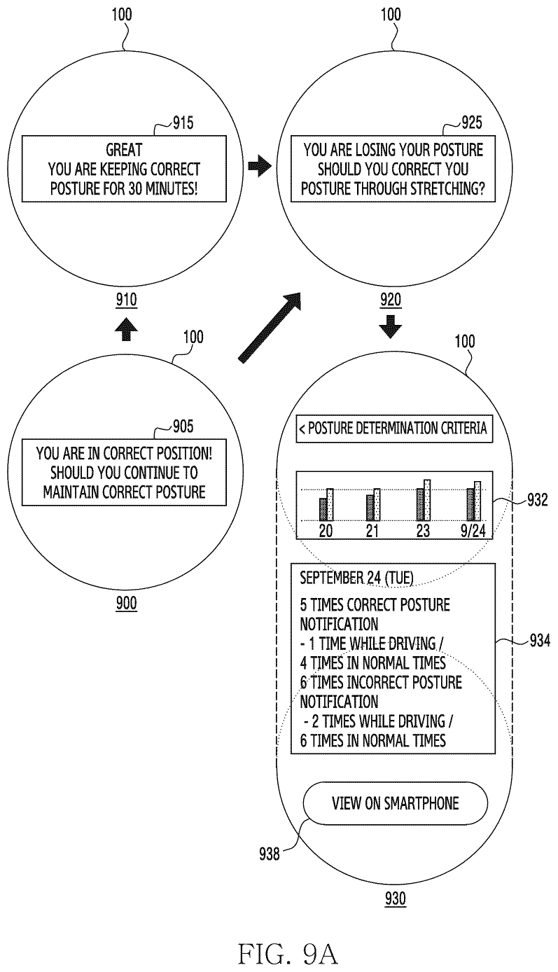

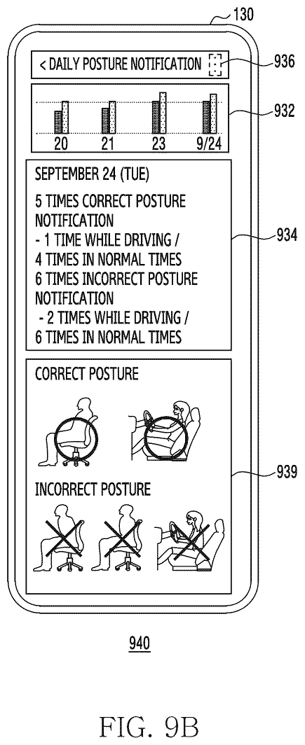

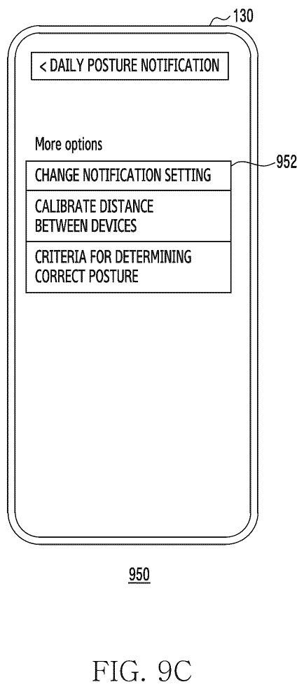

The wearable device 100 may estimate or determine the posture of the user 120 based on identifying the distance between the external electronic device 110 and the wearable device 100. For example, the wearable device 100 may identify a distance between a first part (e.g., a hand) of the user 120 and a second part (e.g., a head) of the user 120 according to the distance. For example, the wearable device 100 may provide the user 120 with a notification based on a state of the posture of the user 120 according to the distance between the wearable device 100 and the external electronic device 110. For example, the wearable device 100 may display a user interface (UI) object (e.g., a first UI object 915 of FIG. 9A) indicating that the posture of the user 120 is correct through a display (e.g., a display 208 of FIG. 2). For example, the wearable device 100 may display another UI object (e.g., a second UI object 925 of FIG. 9A) indicating that the posture of the user 120 is incorrect through the display. For example, the user 120 may correct the posture by identifying the UI object and/or the other UI object. For example, the wearable device 100 may cause a change in the posture of the user 120 by displaying the UI object and/or the other UI object. For example, the wearable device 100 may enhance usability of the wearable device 100 by displaying the UI object and/or the other UI object.

The wearable device 100 may identify the posture of the user 120 through a tilt sensor. For example, the external electronic device 110 may include a gyro sensor or a tilt sensor. For example, the external electronic device 110 may identify a tilt of the external electronic device 110 using the gyro sensor or the tilt sensor. For example, the external electronic device 110 may transmit a signal indicating the identified tilt to the wearable device 100. For example, the wearable device 100 may identify the tilt of the external electronic device 110 by receiving the signal through the communication circuitry (e.g., the communication circuitry 205 of FIG. 2). For example, the wearable device 100 may determine or estimate the posture of the user 120 using the identified tilt and the distance between the wearable device 100 and the external electronic device 110. For example, the wearable device 100 may output a notification through the display (e.g., the display 208 of FIG. 2) based on the determined posture.

For example, the wearable device 100 may identify the distance between the wearable device 100 and the external electronic device 110, and the tilt of the external electronic device 110. For example, the wearable device 100 may determine the posture of the user 120 based on the distance and the tilt being maintained for a threshold time. For example, the wearable device 100 may provide a notification indicating that the posture is correct based on determination that the distance and the tilt are in a range indicating the correct posture for time exceeding the threshold time (e.g., a predefined threshold time). For example, the wearable device 100 may provide a notification indicating that the posture is incorrect based on determination that the distance and the tilt are outside the range for the time exceeding the threshold time (e.g., a predefined threshold time). For example, the wearable device 100 may consume power to identify the distance and the tilt. For example, the wearable device 100 may periodically operate a sensor to identify whether time during which the distance and the tilt are maintained exceeds the threshold time. For example, the wearable device 100 may include an acceleration sensor. For example, the wearable device 100 may identify the distance and the tilt according to a first period based on determination that acceleration of the wearable device 100 identified through the acceleration sensor exceeds threshold acceleration (e.g., a predefined threshold acceleration). For example, the wearable device 100 may identify the distance and the tilt according to a second period longer than the first period based on determination that the acceleration of the wearable device 100 identified through the acceleration sensor is less than the threshold acceleration (e.g., a predefined threshold acceleration). For example, the wearable device 100 may manage power by controlling a period for identifying the tilt and the distance.

For example, the wearable device 100 may include hardware components used to perform or execute the operations. The hardware components are described and exemplified with reference to FIG. 2.

FIG. 2 is a simplified block diagram of an exemplary wearable device.

Referring to FIG. 2, a wearable device 100 may include at least one processor 207, memory 206, communication circuitry 205, and a display 208.

The at least one processor 207 may include a hardware component for processing data using instructions stored in the memory 206. The hardware component for processing data may include a central processing unit (CPU) (e.g., including processing circuitry). The hardware component for processing data may include a graphic processing unit (GPU) (e.g., including the processing circuitry). The hardware component for processing data may include a display processing unit (DPU) (e.g., including the processing circuitry).

The at least one processor 207 may include one or more cores. For example, the at least one processor 207 may have a structure of a multi-core processor such as a dual core, a quad core, or a hexa core.

The memory 206 may include a hardware component for storing data and/or instructions inputted to and/or outputted from the at least one processor 207. The memory 206 may include, for example, volatile memory such as random-access memory (RAM) and/or non-volatile memory such as read-only memory (ROM). The volatile memory may include, for example, at least one of dynamic RAM (DRAM), static RAM (SRAM), cache RAM, and pseudo SRAM (PSRAM). The non-volatile memory may include, for example, at least one of programmable ROM (PROM), erasable PROM (EPROM), electrically erasable PROM (EEPROM), flash memory, a hard disk, a compact disk, and an embedded multimedia card (EMMC).

The communication circuitry 205 may include a hardware component for supporting transmission and/or reception of a signal between the wearable device 100 and an external electronic device. The communication circuitry 205 may include, for example, at least one of a modem, an antenna, and an optic/electronic (O/E) converter. The communication circuitry 205 may support transmission and/or reception of a signal based on various types of protocols, such as Ethernet, local area network (LAN), wide area network (WAN), wireless fidelity (WiFi), Bluetooth, Bluetooth low energy (BLE), Zigbee, long term evolution (LTE), and 5G new radio (NR).

The display 208 may output visualized information. For example, the display 208 may output visualized information to a user according to control of the at least one processor 207. The display 208 may include a hardware component of the wearable device 100 used to display a screen. For example, the display 208 may include light-emitting elements and circuits (e.g., transistors) that control the light-emitting elements to emit light. For example, each of the light-emitting elements may include an organic light emitting diode (OLED) or a micro LED. However, it is not limited thereto. For example, the display 208 may include a liquid crystal display (LCD).

The at least one processor 207 may obtain information associated with a distance between the wearable device 100 and an external electronic device 110 while identifying the external electronic device 110 through communication circuitry 205. For example, the communication circuitry 205 may be used to identify the external electronic device 110. For example, the communication circuitry 205 may be used to receive information from the external electronic device 110. The at least one processor 207 may identify the distance between the wearable device 100 and the external electronic device 110 by using the information. Based on identifying the distance, the at least one processor 207 may identify whether the distance is included in a range set according to a reference distance between the wearable device 100 and the external electronic device 110 that was obtained at a second time point before a first time point at which the information is obtained. The at least one processor 207 may identify time during which the distance has been maintained in a range based on obtaining the distance included in the range for defining a posture of a user 120 wearing the wearable device 100 and the external electronic device 110. By identifying the time exceeding the threshold time (e.g., a predefined threshold time) defined to output a notification associated with the posture, the at least one processor 207 may display a first UI object (e.g., the first UI object 915 of FIG. 9A) indicating that the posture is correct through the display 208. The at least one processor 207 may identify another time during which a distance has been maintained outside the range based on obtaining the distance outside the range for defining the posture of the user 120. By identifying the other time exceeding another threshold time (e.g., another predefined threshold time) defined to output the notification associated with the posture, the at least one processor 207 may display a second UI object (e.g., a second UI object 925 of FIG. 9A) indicating that the posture is incorrect through the display 208. For example, the display 208 may be used to output a notification associated with the distance. For example, the display 208 may be used to display the first UI object and the second UI object.

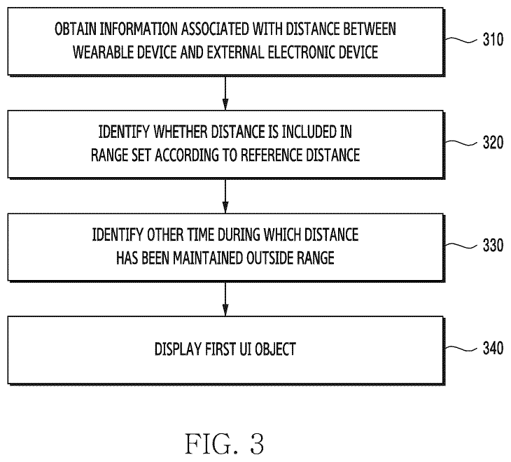

FIG. 3 is a flowchart illustrating an operation of a wearable device that provides a notification based on a distance between the wearable device and an external electronic device. This method may be executed by the wearable device 100 or the at least one processor 207 of the wearable device 100 illustrated in FIG. 2.

Referring to FIG. 3, in an operation 310, the wearable device 100 may obtain information associated with a distance between the wearable device 100 and an external electronic device 110 while identifying the external electronic device 110 through communication circuitry 205. For example, the wearable device 100 may further include a ToF sensor (not illustrated). For example, the wearable device 100 may obtain the information associated with the distance between the wearable device 100 and the external electronic device 110 using the ToF sensor. For example, the wearable device 100 may obtain the information associated with the distance using an RSSI based on the communication circuitry 205. For example, the information may include sensor data. However, it is not limited thereto. For example, the wearable device 100 may receive the information from the external electronic device 110 through the communication circuitry 205. For example, the external electronic device 110 may obtain the information associated with the distance between the wearable device 100 and the external electronic device 110 using the ToF sensor included in the external electronic device 110 or communication circuitry of the external electronic device 110 and then transmit it to the wearable device 100.

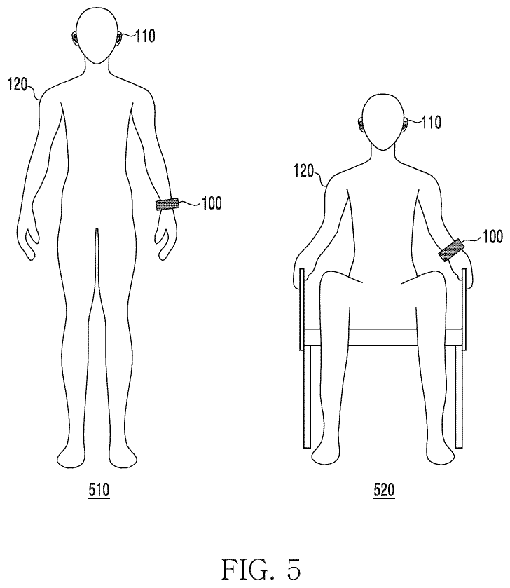

In an operation 320, the wearable device 100 may identify the distance between the wearable device 100 and the external electronic device 110 by using the information associated with the distance between the wearable device 100 and the external electronic device 110. For example, the wearable device 100 may identify the distance by analyzing the information. However, it is not limited thereto. For example, the wearable device 100 may identify the distance indicated by the information. Based on identifying the distance, the wearable device 100 may identify whether the distance is included in a range set according to a reference distance between the wearable device 100 and the external electronic device 110 that was obtained at a second time point before a first time point at which the information is obtained. For example, the wearable device 100 may obtain the reference distance between the wearable device 100 and the external electronic device 110 before identifying the distance. For example, the wearable device 100 may obtain the reference distance between the wearable device 100 and the external electronic device 110 while the user 120 is in a specific posture (e.g., an attention posture or a sitting posture). For example, the reference distance may be used to set a range for defining the posture of the user 120. For example, the reference distance may be obtained before obtaining the distance. For example, the reference distance may be obtained at the second time point before the first time point at which the information is obtained. For example, the range may be set to define a first posture of the user 120 wearing the wearable device 100 and the external electronic device 110. Obtaining the reference distance will be described later with reference to FIG. 5.

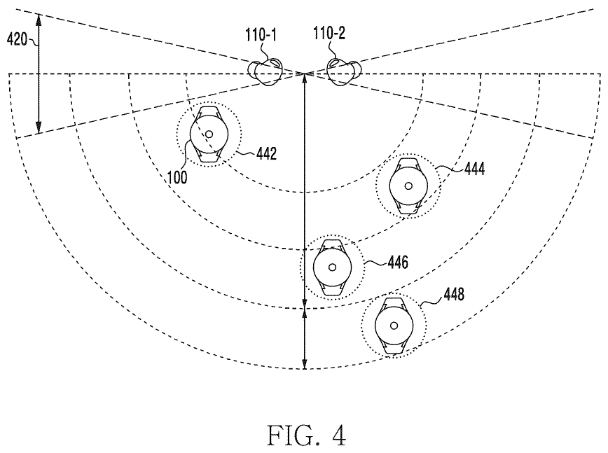

According to an embodiment, based on obtaining a distance included in the range for defining the posture of the user 120 wearing the wearable device 100 and the external electronic device 110, the wearable device 100 may identify a time during which the distance has been maintained in the range. For example, the wearable device 100 may determine the posture of the user 120 based on determination that the distance is included in the range. For example, the determination of the posture is described and exemplified in more detail with reference to FIG. 4.

FIG. 4 illustrates an exemplary operation of a wearable device identifying a tilt of an external electronic device and a position of the wearable device.

Referring to FIG. 4, a wearable device 100 may determine a posture of a user 120 according to a ratio of a distance between the wearable device 100 and an external electronic device 110, and a reference distance between the wearable device 100 and the external electronic device 110. The reference distance between the wearable device 100 and the external electronic device 110 is measured or determined in advance. The reference distance is a value. The distance or current distance between the wearable device 100 and the external electronic device 110 is recently measured or determined, for example, in near real time or real time. For example, an external electronic device 115 may be an example of the external electronic device 110. For example, an external electronic device 105 may be an example of the wearable device 100. For example, the wearable device 100 may define a range for determining that the posture of the user 120 is a correct posture using the reference distance. For example, the wearable device 100 may define a first range for determining that the posture of the user 120 is correct based on the reference distance. For example, the first range may be distinguished according to a specific ratio (e.g., 80%) of the reference distance. For example, a criterion for determining to be included in the first range may be the specific ratio of the reference distance. For example, the wearable device 100 may define a second range, a third range, and a fourth range for determining that the posture of the user 120 is incorrect based on the reference distance. For example, the second range may be distinguished according to a specific ratio (e.g., 60%) of the reference distance. For example, the first range may be 80% of the reference distance to 100% of the reference distance. However, it is not limited thereto. For example, the first range may be 80% or more of the reference distance. For example, the second range may be 60% of the reference distance to 80% of the reference distance. For example, the third range may be 40% of the reference distance to 60% of the reference distance. For example, the fourth range may be 0% to 40% of the reference distance. For example, the wearable device 100 may provide a notification differently based on determination that the distance between the wearable device 100 and the external electronic device 110 is in a specific range.

For example, the wearable device 100 may obtain the distance at a first time point. For example, the wearable device 100 may obtain the reference distance at a second time point before the first time point.

For example, in a case that the wearable device 100 is positioned at a position 442, the distance may be included in the fourth range. For example, in a case that the wearable device 100 is positioned at a position 444, the distance may be included in the third range. For example, in a case that the wearable device 100 is positioned at a position 446, the distance may be included in the second range. For example, in a case that the wearable device 100 is positioned at a position 448, the distance may be included in the first range. For example, the wearable device 100 may provide the notification differently based on a range in which the distance is included. For example, the wearable device 100 may provide a vibration notification at a first intensity based on determination that the distance is included in the second range. For example, the wearable device 100 may provide a vibration notification at a second intensity stronger than the first intensity based on determination that the distance is included in the third range. For example, the wearable device 100 may identify a time during which the distance has been maintained in the first range based on obtaining the distance included in the first range. For example, the wearable device 100 may control an actuator to provide the vibration notification at the first intensity by identifying the time exceeding the threshold time. For example, the wearable device 100 may identify another time during which the distance has been maintained outside the first range based on obtaining the distance that is not included in the first range (or outside the first range). For example, the wearable device 100 may control the actuator to provide the vibration notification at the second intensity different from the first intensity by identifying the other time exceeding another threshold time. For example, a range not included in the first range may include at least one of the second range, the third range, and/or the fourth range, which can have a vibration notification as a vibration intensity greater than the first intensity.

According to an embodiment, the wearable device 100 may change an intensity of a vibration notification based on the time during which the distance has been maintained. For example, the wearable device 100 may control the actuator to provide the vibration notification at the first intensity based on a determination that the time during which the distance has been maintained outside the first range exceeds a first threshold time. For example, the wearable device 100 may control the actuator to provide the vibration notification at the second intensity stronger than the first intensity based on determination that the time exceeds a second threshold time longer than the first threshold time. For example, the wearable device 100 may identify the time during which the distance has been maintained outside the first range. For example, the wearable device 100 may control the actuator to provide the vibration notification at the first intensity based on identifying the time exceeding the threshold time. For example, the wearable device 100 may control the actuator to provide the vibration notification at the second intensity different from (or greater than) the first intensity based on identifying another time exceeding the time during which the distance has been maintained outside the first range.

The wearable device 100 may determine the posture of the user 120 by further using a tilt of the external electronic device 110. For example, the external electronic device 110 may include a gyro sensor or a tilt sensor. For example, the external electronic device 110 may identify the tilt of the external electronic device 110 using the gyro sensor or the tilt sensor. For example, when obtaining the reference distance between the wearable device 100 and the external electronic device 110, the wearable device 100 may obtain a reference tilt of the external electronic device 110. For example, the wearable device 100 may obtain the reference tilt at the second time point. For example, the wearable device 100 may identify or obtain the tilt of the external electronic device 110 at the first time point after the second time point. The reference tilt can be determined in advance and stored in memory 206 of the wearable device 100. The reference tilt is related to a distance between the head of the user 120 to the electronic wearable device 100, such that as the head of the user 120 tilts forward (e.g., downward) the distance decreases between the external electronic device 110 and the wearable device 100. Similarly, the reference tilt is related to a distance between the head of the user 120 to the electronic wearable device 100, such that as the head of the user 120 tilts backward the distance increases between the external electronic device 110 and the wearable device 100. For example, the wearable device 100 may obtain a tilt 420 by using each of an external electronic device 110-1 and an external electronic device 110-2. For example, each of the external electronic device 110-1 and the external electronic device 110-2 may obtain the tilt 420 using the tilt sensor or the gyro sensor. For example, the external electronic device 110 may transmit a signal indicating the tilt 420 to the wearable device 100. The external electronic device 110 may include the external electronic device 110-1 and/or the external electronic device 110-2. For example, the external electronic device 110-1 and/or the external electronic device 110-2 may transmit the signal indicating the tilt 420 to the wearable device 100. For example, the wearable device 100 may identify the tilt 420 of the external electronic device 110 based on receiving the signal indicating the tilt 420 from the external electronic device 110 through communication circuitry 205.

For example, the wearable device 100 may determine that the posture of the user 120 is correct based on a determination that the tilt 420 is in a tilt range set by the reference tilt. For example, the wearable device 100 may determine that the posture of the user 120 is incorrect based on a determination that the tilt 420 is outside the tilt range set by the reference tilt. For example, the wearable device 100 may determine that the posture of the user 120 is correct based on a determination that the tilt 420 of the external electronic device 110 is in the tilt range and the distance between the wearable device 100 and the external electronic device 110 is in the first range. For example, based on determining that the posture of the user 120 is correct for a specific time, the wearable device 100 may display a first UI object (e.g., a first UI object 915 of FIG. 9A) indicating that the posture of the user 120 is correct through a display 208. For example, the wearable device 100 may determine that the posture of the user 120 is incorrect, based on a determination that the tilt 420 of the external electronic device 110 is not in the tilt range or the distance between the wearable device 100 and the external electronic device 110 is not in the first range (or determination that the distance is outside the first range). For example, based on the determination that the posture of the user 120 is incorrect for a specific time, the wearable device 100 may display a second UI object (e.g., a second UI object 925 of FIG. 9A) indicating that the posture of the user 120 is incorrect through the display 208.

According to an embodiment, the wearable device 100 may identify a tilt using a wrist-wearable electronic device (e.g., the wearable device 100) or a hand-wearable electronic device (e.g., the external electronic device 105). For example, the wearable device 100 may determine a reference tilt of head-wearable electronic devices (e.g., the external electronic device 110 and the external electronic device 115) differently from a first other reference tilt of the wrist-wearable electronic device or a second other reference tilt of the hand-wearable electronic device. For example, the wearable device 100 may determine the posture of the user 120 as a correct posture or an incorrect posture according to a range (e.g., −30 degrees to +30 degrees, when a case in which a direction perpendicular to the display 208 of the wearable device 100 and a direction of gravity are parallel is defined as 0 degrees) set by the first other reference tilt. For example, the wearable device 100 may determine the posture of the user 120 as the correct posture or the incorrect posture according to a range (e.g., −30 degrees to +30 degrees, when a case in which a direction in which a finger of the user 120 wearing the external electronic device 105 directs and the direction of gravity are perpendicular is defined as 0 degrees, or when a case in which a central axis of the external electronic device 105 and the direction of gravity are perpendicular is defined as 0 degrees) set by the second other reference tilt.

For example, information associated with the distance between the wearable device 100 and the external electronic device 110 may further include (additional) information associated with the tilt of the external electronic device 110. For example, the wearable device 100 may identify the tilt 420 of the external electronic device 110 using the additional information. Based on identifying the tilt 420, the wearable device 100 may further identify whether the tilt 420 is included in the tilt range set according to the reference tilt of the external electronic device 110 that was obtained at the second time point before the first time point when the information is obtained. For example, the wearable device 100 may identify the time during which the tilt 420 has been maintained in a tilt range based on obtaining the tilt 420 included in the tilt range for defining the posture of the user 120.

According to an embodiment, the wearable device 100 may change a period of obtaining the distance and the tilt based on an identified threshold acceleration. For example, the wearable device 100 may further include an acceleration sensor. For example, the wearable device 100 may identify, measure, or obtain acceleration of the wearable device 100 using the acceleration sensor. For example, the wearable device 100 may change a period of obtaining the distance or the tilt 420 based on the acceleration of the wearable device 100. For example, the wearable device 100 may obtain the distance between the wearable device 100 and the external electronic device 110 and the tilt 420 of the external electronic device 110 according to a first period. For example, a ToF sensor may be periodically enabled by the wearable device 100. For example, the wearable device 100 may identify whether the acceleration of the wearable device 100 exceeds the threshold acceleration (e.g., a predefined threshold acceleration). For example, the wearable device 100 may obtain or identify the distance and the tilt according to a second period longer than the first period based on determination that the acceleration of the wearable device 100 is less than the threshold acceleration. For example, the wearable device 100 may obtain or identify the distance and the tilt according to the first period shorter than the second period based on determination that the acceleration of the wearable device 100 exceeds the threshold acceleration.

For example, the wearable device 100 may identify acceleration of the external electronic device 110 at least based on an acceleration sensor included in the external electronic device 110 using the information associated with the distance between the wearable device 100 and the external electronic device 110. For example, the acceleration sensor may be periodically enabled according to the first period. For example, based on identifying the acceleration is less than the threshold acceleration, the wearable device 100 may transmit a signal instructing to change a period of the acceleration sensor to the second period longer than the first period to the external electronic device 110 through the communication circuitry 205. For example, based on identifying the acceleration exceeds the threshold acceleration, the wearable device 100 may transmit another signal instructing to maintain the period of the acceleration sensor as the first period to the external electronic device 110 through the communication circuitry 205.

For example, based on a determination that the acceleration of the wearable device 100 is less than the threshold acceleration, the wearable device 100 may transmit a signal instructing to transmit data indicating the tilt 420 according to the second period to the external electronic device 110 through the communication circuitry 205. For example, based on a determination that the acceleration of the wearable device 100 exceeds the threshold acceleration, the wearable device 100 may transmit a signal instructing to transmit data indicating the tilt 420 according to the first period to the external electronic device 110 through the communication circuitry 205.

For example, the external electronic device 110 may receive data indicating the acceleration of the wearable device 100 from the wearable device 100. For example, based on a determination that the acceleration of the wearable device 100 identified from the data is less than the threshold acceleration, the external electronic device 110 may transmit data indicating the tilt 420 of the external electronic device 110 to the wearable device 100 according to the second period. For example, based on a determination that the acceleration of the wearable device 100 identified from the data exceeds the threshold acceleration, the external electronic device 110 may transmit the data indicating the tilt 420 of the external electronic device 110 to the wearable device 100 according to the first period.

For example, the wearable device 100 may receive the information from the external electronic device 110 according to the first period through the communication circuitry 205 based on a determination that the acceleration of the external electronic device 110 obtained through the acceleration sensor included in the external electronic device 110 exceeds the threshold acceleration. For example, the external electronic device 110 may determine a period of the information to be transmitted to the wearable device 100 based on the acceleration obtained through the acceleration sensor. For example, the wearable device 100 may receive the information from the external electronic device 110 through the communication circuitry 205 according to the second period longer than the first period based on a determination that the acceleration of the external electronic device 110 obtained through the acceleration sensor is less than the threshold acceleration.

According to an embodiment, the external electronic device 110 may further include an acceleration sensor. For example, based on determination that the acceleration of the external electronic device 110 identified using the acceleration sensor exceeds the threshold acceleration, the external electronic device 110 may transmit data indicating the tilt of the external electronic device 110 to the wearable device 100 according to the first period. For example, the external electronic device 110 may transmit the data to the wearable device 100 according to the second period longer than the first period based on determination that the acceleration is less than the threshold acceleration.

According to an embodiment, the wearable device 100 may further include a rechargeable battery (not illustrated). For example, the wearable device 100 may change the period of obtaining the distance and the tilt 420 based on a state of charge (SoC) of the rechargeable battery. For example, the wearable device 100 may obtain the distance and the tilt 420 of the external electronic device 110 according to the first period based on a determination that the SoC exceeds a threshold SoC. For example, the wearable device 100 may obtain the distance and the tilt 420 of the external electronic device 110 according to the second period longer than the first period based on determination that the SoC is less than the threshold SoC. For example, the wearable device 100 may reduce power to obtain the distance and the tilt 420 by changing the period for obtaining the distance and the tilt 420 based on the acceleration and the SoC. For example, the wearable device 100 may manage the power to obtain the distance and the tilt 420 by changing the period.

In the above, an operation of providing a notification based on the distance between the wearable device 100 and the external electronic device 110 is described, but an embodiment of the present disclosure is not limited thereto. For example, an external electronic device 130 may provide a notification based on a distance between the external electronic device 130 and the external electronic device 110. For example, the external electronic device 130 may provide a notification based on a distance between the external electronic device 130 and the external electronic device 115. For example, the external electronic device 130 may obtain distances from each of the external electronic device 110 and the external electronic device 115. For example, the external electronic device 115 may identify a distance between a part (e.g., a hand) of the user 120 and the external electronic device 115 based on obtaining an image including a depth value through a camera included in the external electronic device 115. For example, the external electronic device 115 may identify whether the distance is included in a range for defining the posture of the user by identifying the distance between the part of the user 120 and the external electronic device 115 using the image. For example, the external electronic device 115 may identify the distance between the part of the user 120 and the external electronic device 115 by providing the image to a trained model. For example, the external electronic device 115 may identify the distance by performing object detection using the trained model. For example, the external electronic device 115 may provide a notification based on the identification.

Referring back to FIG. 3, in the operation 320, the wearable device 100 may identify a time during which the distance between the wearable device 100 and the external electronic device 110 has been maintained in the first range.

For example, based on obtaining a distance included in the first range for defining the posture of the user 120, the wearable device 100 may display a first UI object (e.g., a first UI object 915 of FIG. 9A) indicating that the posture is correct through the display 208, by identifying the time exceeding threshold time defined to output a notification associated with the posture. For example, the wearable device 100 may identify a time during which the distance has been included in a first range. For example, the wearable device 100 may display a UI object associated with the posture through the display 208, by identifying the time exceeding the threshold time. However, it is not limited thereto. For example, the wearable device 100 may control an actuator (not illustrated) to provide a vibration notification together with the UI object. For example, the wearable device 100 may control a speaker (not illustrated) to output an audio signal together with the UI object.

Based on identifying the time exceeding the threshold time, the wearable device 100 may display the first UI object (e.g., the first UI object 915 of FIG. 9A) indicating that the posture of the user 120 is the first posture through the display 208. For example, the first posture may be referred to as a correct posture. For example, the first posture may be described as a posture corresponding to the first range described in FIG. 4.

In an operation 330, the wearable device 100 may identify another time during which the distance has been maintained outside the first range based on obtaining the distance outside the first range for defining the posture of the user 120. For example, the wearable device 100 may identify the other time when the distance outside the first range is obtained.

In an operation 340, based on obtaining the distance outside the first range for defining the posture of the user 120, the wearable device 100 may display a second UI object (e.g., a second UI object 925 of FIG. 9A) indicating that the posture is incorrect through the display 208, by identifying the other time exceeding another threshold time defined to output a notification associated with the posture. For example, the wearable device 100 may display the second UI object (e.g., the second UI object 925 of FIG. 9A) indicating that the posture of the user 120 corresponds to a second posture through the display 208 based on identifying the other time exceeding the other threshold time. For example, the second posture may be referred to as an incorrect posture. For example, the second posture may correspond to each of the second range, the third range, and the fourth range described in FIG. 4.

An operation of providing the notification based on the distance between the wearable device 100 and the external electronic device 110 has been described above, but an embodiment is not limited thereto. For example, based on the distance between the wearable device 100 and the external electronic device 110, the external electronic device 130 may provide a notification. For example, each of the wearable device 100 and the external electronic device 110 may transmit a signal indicating the distance to the external electronic device 130. For example, the external electronic device 130 may provide the notification based on the received signal.

For example, the wearable device 100 may obtain a reference distance to set the first range. For example, obtaining the reference distance is described and exemplified in more detail with reference to FIG. 5.

FIG. 5 illustrates an exemplary operation of a wearable device for obtaining a reference distance between the wearable device and an external electronic device.

Referring to FIG. 5, a state 510 may be described as a state of obtaining a reference distance in a state in which a user 120 is standing. A state 520 may be described as a state of obtaining the reference distance in a state in which the user 120 is sitting. For example, a wearable device 100 may obtain a reference distance between the wearable device 100 and an external electronic device 110 using a ToF sensor or communication circuitry 205. For example, the reference distance may be used to determine ranges for defining a posture of the user 120. For example, a first range indicating a correct posture may be described as a range between a first ratio (e.g., 80%) and a second ratio (e.g., 100%) of the reference distance.

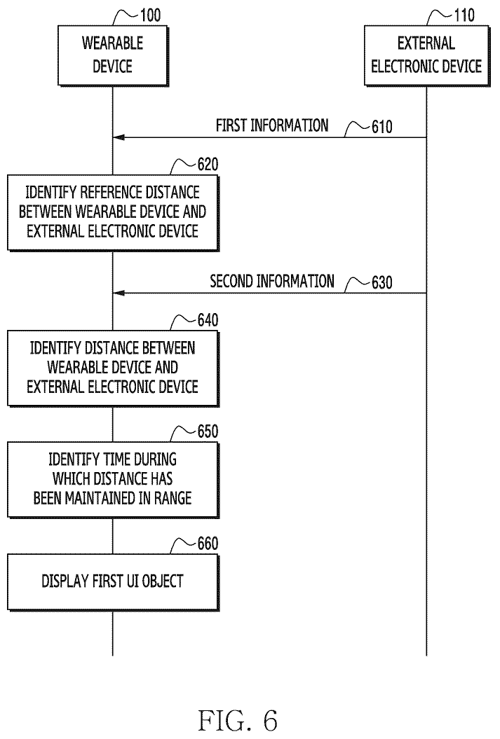

For example, the wearable device 100 may obtain the longest distance between the external electronic device 110 and the wearable device 100 in the state in which the user 120 is standing. For example, in the state in which the user 120 is sitting, the wearable device 100 may obtain a reference distance for determining the posture while the user 120 is working in a sitting state. For example, the wearable device 100 may obtain the reference distance by using information associated with a distance between the external electronic device 110 and the wearable device 100. For example, the wearable device 100 may obtain the information from the external electronic device 110. For example, the information obtained from the external electronic device 110 is described and exemplified in more detail with reference to FIG. 6.

FIG. 6 illustrates an example of operations executed in a wearable device and an external electronic device.

Referring to FIG. 6, in an operation 610, an external electronic device 110 may transmit first information to a wearable device 100. For example, the wearable device 100 may receive the first information from the external electronic device 110 through communication circuitry 205. For example, the first information may be information indicating a reference distance between the wearable device 100 and the external electronic device 110 obtained from the external electronic device 110. For example, the external electronic device 110 may obtain the first information through a ToF sensor or an RSSI based on communication circuitry.

In an operation 620, the wearable device 100 may identify the reference distance between the wearable device 100 and the external electronic device 110 using the first information.

In an operation 630, the external electronic device 110 may transmit second information indicating a distance between the external electronic device 110 and the wearable device 100 to the wearable device 100. For example, the wearable device 100 may receive the second information from the external electronic device 110 through the communication circuitry 205. For example, the second information may be information obtained at a second time point after the first time point when the first information was obtained. For example, the operation 630 may correspond to the operation 310 of FIG. 3.

In an operation 640, the wearable device 100 may identify the distance between the wearable device 100 and the external electronic device 110 using the second information. For example, the operation 640 may correspond to the operation 320 of FIG. 3.

In an operation 650, the wearable device 100 may identify a time during which the distance has been maintained in a first range.

In an operation 660, the wearable device 100 may display a first UI object (e.g., a first UI object 915 of FIG. 9A). For example, the wearable device 100 may display the first UI object through a display 208 according to the determination that the time during which the distance has been maintained in the first range exceeds a threshold time.

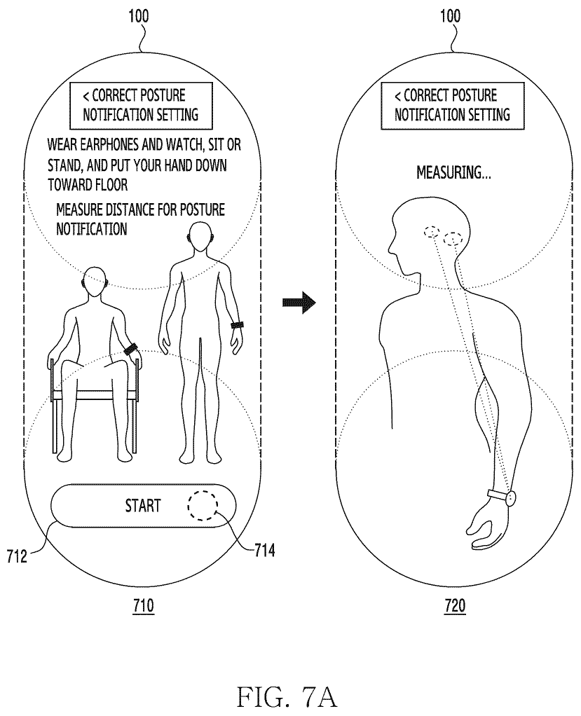

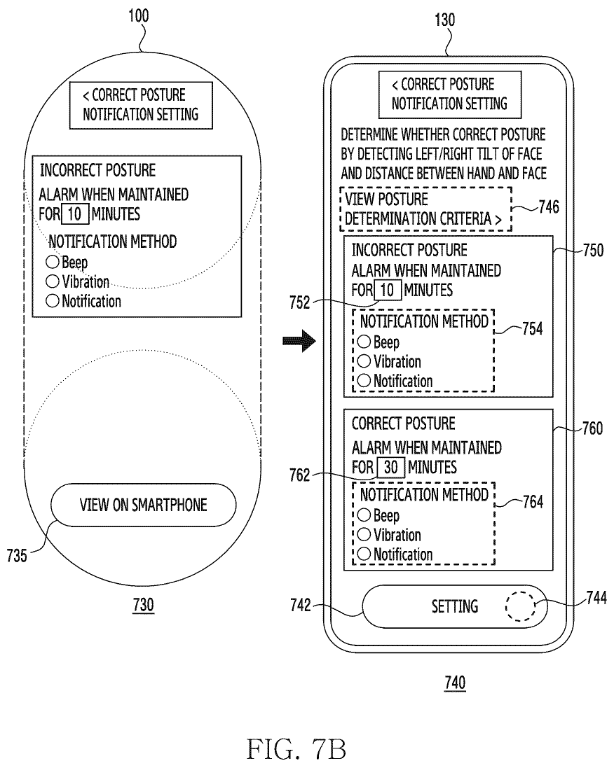

For example, when obtaining the reference distance, the wearable device 100 may display a screen associated with obtaining the reference distance through the display 208. For example, the wearable device 100 may start obtaining the reference distance based on receiving a user input with respect to the screen. For example, after obtaining the reference distance, the wearable device 100 may display another screen for a setting to provide a notification through the display 208. For example, the display of the screen and the other screen is described and exemplified in more detail with reference to FIGS. 7A and 7B.

FIGS. 7A and 7B illustrate an exemplary operation of a wearable device displaying a screen for a setting for providing a notification.

Referring to FIG. 7A, a state 710 may be described in a state in which a screen for obtaining the reference distance is displayed. For example, based on receiving a user input for executing a software application for monitoring a posture of a user 120, a wearable device 100 may execute the software application. For example, the wearable device 100 may display a screen associated with obtaining the reference distance based on executing the software application. For example, when executing the software application before obtaining information associated with a distance between the wearable device 100 and an external electronic device 110, the wearable device 100 may display a screen indicating a reference posture provided by the software application through a display 208. For example, the wearable device 100 may display the screen including text indicating the posture (e.g., a standing posture or a sitting posture) of the user 120 through the display 208. For example, the screen may include a visual object 712. For example, the visual object 712 may be a visual object that causes the reference distance between the wearable device 100 and the external electronic device 110 to be measured. For example, the wearable device 100 may obtain the reference distance between the wearable device 100 and the external electronic device 110 based on receiving a user input 714 for the visual object 712. In the state 710, a visual object representing the user 120 wearing the external electronic device 110 may be displayed, but in a case that the user 120 wears an external electronic device 115, another visual object representing the user 120 wearing the external electronic device 115 may be displayed.

A state 720 may be described as a state of obtaining the reference distance. For example, the wearable device 100 may switch a state of the wearable device 100 from the state 710 to the state 720 based on receiving the user input 714. For example, the wearable device 100 may obtain the reference distance between the wearable device 100 and the external electronic device 110 in the state 720. For example, the wearable device 100 may obtain the reference distance using a ToF technique or an RSSI. However, it is not limited thereto.

Referring to FIG. 7B, a state 730 may be described as a state in which a screen associated with a method of providing a notification is displayed. For example, while displaying the screen, the wearable device 100 may determine a method of a notification and/or threshold time based on receiving a user input. For example, the method of the notification may include at least one of an audio signal indicating a specific audio (e.g., a beep), a vibration notification, and/or a UI object. For example, the threshold time may be described as the time, during which the distance between the wearable device 100 and the external electronic device 110 and a tilt of the external electronic device 110 have been maintained, required to provide the notification. For example, the wearable device 100 may cause the external electronic device to display a screen for setting the method of the notification and the threshold time through an external electronic device 130 (e.g., a smartphone) based on receiving a user input for a visual object 735 in the state 730. For example, when executing the software application, the wearable device 100 may display the screen for setting the threshold time and the method of the notification through the display 208. For example, based on receiving a user input for the screen, the wearable device 100 may control an actuator to provide a vibration notification together with outputting an audio signal (e.g., a beep signal) corresponding to the notification through a speaker by identifying the time exceeding the threshold time. For example, when identifying the time exceeding the threshold time based on receiving the user input for the screen, the wearable device 100 may perform at least one of displaying a UI object (e.g., the first UI object 915 of FIG. 9A) through the display 208, outputting the audio signal corresponding to the notification through the speaker, and/or controlling the actuator to provide the vibration notification. For example, the wearable device 100 may receive a user input for selecting the at least one.

For example, when executing a software application for monitoring a body posture, the wearable device 100 may display the screen for setting the threshold time through the display 208. For example, the wearable device 100 may determine the threshold time based on receiving a user input for setting the threshold time.

A state 740 may be described as a state in which a screen for setting the method of providing the notification through a display of the external electronic device 130 is displayed. For example, the external electronic device 130 may display a visual object 750 that may control a setting for an incorrect posture. For example, the visual object 750 may include a visual object 752 for controlling the threshold time. For example, the external electronic device may determine threshold time associated with providing a notification based on receiving a user input for the visual object 752. For example, the visual object 750 may include a visual object 754 for controlling the method of the notification. For example, the external electronic device may change the method of the notification based on receiving another user input for the visual object 754.

For example, the external electronic device 130 may display a visual object 760 for changing a setting for a correct posture. For example, the external electronic device may display the visual object 760 including a visual object 762 for setting threshold time linked to the correct posture. For example, a visual object 764 for changing a method of a notification for the correct posture may be included in the visual object 760.

For example, the external electronic device 130 may cause the wearable device 100 to identify the distance between the wearable device 100 and the external electronic device 110 and the tilt of the external electronic device 110 based on receiving a user input 744 for a visual object 742. For example, the visual object 742 may be a visual object for causing a notification to be provided according to the distance and the tilt. For example, the wearable device 100 may provide a notification according to the method and the threshold time set in the state 740 based on the user input 744.

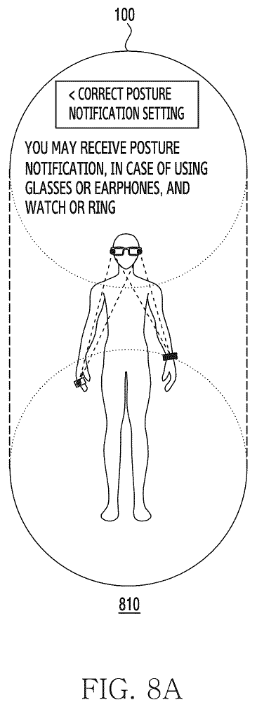

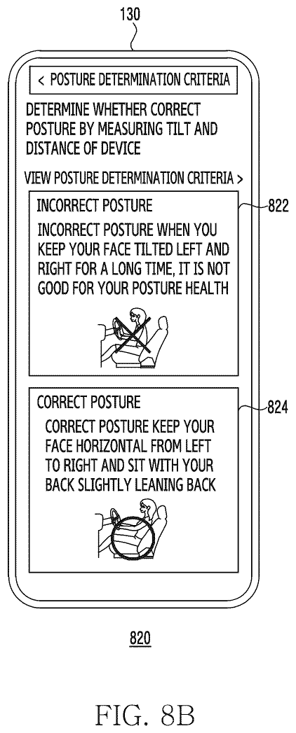

The external electronic device 130 may display a visual object 746 in the state 740. For example, the visual object 746 may be described as a visual object for guiding the correct posture of the user 120. For example, the external electronic device 130 may cause the wearable device 100 to display a screen guiding the correct posture of the user 120 based on receiving a user input for the visual object 746. For example, the wearable device 100 may display another screen on the display 208 indicating that the user 120 may not receive a notification for the posture of the user 120 based on identifying that the user 120 does not wear a head-wearable electronic device (e.g., the external electronic device 110) or the wearable device 100. For example, the display of the screen and the other screen is described and exemplified in more detail with reference to FIGS. 8A and 8B.

FIGS. 8A and 8B illustrate an exemplary operation of a wearable device displaying a screen associated with a notification.

Referring to FIG. 8A, a state 810 may be described as a state in which a screen indicating that a notification based on a posture of a user 120 may not be provided is displayed. For example, the wearable device 100 may identify, through a sensor (e.g., a photoplethysmography (PPG) sensor), whether it is in a state of being worn by the user 120. For example, the wearable device 100 may identify whether the external electronic device 110 is worn by the user 120 based on a connection with the external electronic device 110 through communication circuitry 205. For example, according to a determination that the external electronic device 110 or the wearable device 100 is not worn by the user 120, the wearable device 100 may display a screen indicating that a notification based on a distance between the wearable device 100 and the external electronic device 110 is not provided. For example, the state 810 may be described as a state of receiving the user input 714 for the visual object 712 of the state 710 in a state that the wearable device 100 or the external electronic device 110 is not worn by the user 120.

Referring to FIG. 8B, the state 820 may be described as a state of displaying another screen for a criterion for determining a posture. For example, the state 820 may be described as a state change according to a reception of the user input for the visual object 746 in the state 740. For example, the wearable device 100 may display a visual object 822 that guides (or identifies) an incorrect posture and a visual object 824 that guides a correct posture through the display 208. For example, the user 120 may maintain a correct posture by recognizing the visual object 822 and the visual object 824. For example, the wearable device 100 may cause the correct posture of the user 120 by displaying the visual object 822 and the visual object 824.

After receiving the user input 744 in the state 740, the wearable device 100 may provide a notification (e.g., a beep, a vibration notification, or a display of a UI object) based on the distance between the wearable device 100 and the external electronic device 110. Providing the notification is described and exemplified in more detail with reference to FIGS. 9A to 9C.

FIGS. 9A to 9C illustrate an exemplary operation of a wearable device displaying a UI object.

Referring to FIG. 9A, a state 900 may be described as a state in which a UI object 905 indicating that a posture of a user 120 is changed is displayed. For example, a wearable device 100 may display the UI object 905 indicating that the posture of the user 120 changes from an incorrect posture to a correct posture through a display 208. However, it is not limited thereto. For example, the wearable device 100 may provide a notification that causes movement of the user 120 based on the movement of the user 120 not being identified for a specific time (e.g., 50 minutes). For example, after providing the notification, the wearable device 100 may display the UI object 905 indicating that the posture of the user 120 is correct through the display 208 based on identifying that the posture of the user 120 is changed to the correct posture.

The state 910 may be described as a state in which a first UI object 915 indicating that the posture of the user 120 is correct is displayed. A state 920 may be described as a state in which a second UI object 925 indicating that the posture of the user 120 is incorrect is displayed. For example, the wearable device 100 may identify whether a distance between the wearable device 100 and an external electronic device 110 obtained using information is included in a first range. For example, when the time during which the distance has been included in the first range exceeds threshold time, the wearable device 100 may display the first UI object 915 through the display 208. For example, when the time during which the distance has been included in the first range exceeds the threshold time, the wearable device 100 may display the first UI object 915 indicating that the posture of the user 120 is a first posture through the display 208. For example, the first posture may be referred to as the correct posture. For example, the first UI object 915 may indicate that the posture of the user 120 is the first posture.

The state 920 may be described as a state in which the second UI object 925 indicating that a state of the user 120 is incorrect is displayed. For example, when another time during which the distance has not been included in the first range exceeds another threshold time, the wearable device 100 may display the second UI object 925 through the display 208. For example, when the other time during which the distance has not been included in the first range exceeds the other threshold time after displaying the first UI object 915, the wearable device 100 may cease displaying the first UI object 915 and may display the second UI object 925. For example, when the other time during which the distance has not been included in the first range exceeds the other threshold time after displaying the first UI object 915, the wearable device 100 may change or switch an object displayed through the display 208 from the first UI object 915 to the second UI object 925. For example, when the other time during which the distance has not been included in the first range exceeds the other threshold time, the wearable device 100 may display the second UI object 925 indicating that the posture of the user 120 is a second posture through the display 208. For example, the second UI object 925 may be referred to as the incorrect posture. For example, the second UI object 925 may indicate that the posture of the user 120 is the second posture.

According to an embodiment, while displaying the second UI object 925, the wearable device 100 may obtain other information associated with the distance between the wearable device 100 and the external electronic device 110. For example, the wearable device 100 may obtain the other information using a ToF sensor. For example, the wearable device 100 may obtain the other information from the external electronic device 110. For example, the wearable device 100 may cease displaying the second UI object 925 based on identifying that a distance indicated by the other information is included in the first range. For example, the wearable device 100 may switch or change a UI object displayed through the display 208 from the second UI object to the first UI object 915 based on identifying that the distance indicated by the other information is included in the first range. For example, the wearable device 100 may display the first UI object 915 through the display 208 based on identifying that the distance indicated by the other information is included in the first range. For example, after displaying the first UI object 915, the wearable device 100 may display the second UI object 925 based on satisfying a condition (e.g., the other time during which the distance between the wearable device 100 and the external electronic device 110 has not been included in the first range exceeds the other threshold time) for displaying the second UI object 925. For example, after displaying the second UI object 925, the wearable device 100 may display the first UI object 915 based on satisfying a condition (e.g., the time during which the distance between the wearable device 100 and the external electronic device 110 has been included in the first range exceeds the threshold time) for displaying the first UI object 915.

The state 900 may be described as a state in which the UI object 905 is displayed in a case that the posture of the user 120 is changed from the incorrect posture to the correct posture. For example, after displaying the second UI object 925, the wearable device 100 may display the UI object 905 based on identifying that the posture of the user 120 is changed to the correct posture. Referring to FIG. 9A, an operation of switching from the state 900 to the state 920 is illustrated, but an embodiment is not limited thereto. For example, a state of the wearable device 100 may be changed from the state 920 to the state 900. For example, the wearable device 100 may display the UI object 905 based on identifying that the distance is (now) included in the first range after a time during which the distance has not been included in the first range exceeds the threshold time. For example, the wearable device 100 may display the UI object 905 based on identifying that the distance is included in the first range while displaying the second UI object 925 through the display 208 or after displaying the second UI object 925. For example, when the time during which the distance has been included in the first range exceeds the threshold time after displaying the second UI object 925, the wearable device 100 may cease displaying the second UI object 925 and may display the UI object 905. For example, when the time during which the distance has been included in the first range exceeds the threshold time after displaying the second UI object 925, the wearable device 100 may change or switch the object displayed through the display 208 from the second UI object 925 to the UI object 905. For example, the wearable device 100 may record the time during which the distance has been included in the first range when the distance is included within the first range after the distance is not included in the first range for time exceeding the threshold time. The wearable device 100 may display the UI object 905 when the time during which the distance has been included in the first range exceeds the threshold time after the distance is included in the first range again.

A state 930 may be described as a screen on which a statistic of a notification for the posture of the user 120 is displayed. For example, the wearable device 100 may display a visual object 932 representing a graph indicating that the number of times when each of the first UI objects 915 and the second UI object 925 is displayed for each day through the display 208. For example, the wearable device 100 may display a visual object 934 representing a statistic indicating a case (or the number of times) that each of the first UI object 915 and the second UI object 925 is displayed during a day through the display 208. For example, the visual object 934 may represent a statistic for each of a case that the user 120 is in a driving state and a case that the user 120 is not in the driving state. For example, the wearable device 100 may display a visual object 938 for displaying the screen through a display of an external electronic device 130. For example, the wearable device 100 may cause the external electronic device 130 to display the screen through the display of the external electronic device 130 based on receiving a user input for the visual object 938.

Referring to FIG. 9B, a state 940 may be described as a state in which a screen for a statistic of a notification is displayed through the external electronic device 130. For example, the external electronic device 130 may display the visual object 932 and the visual object 934. For example, the external electronic device 130 may further display a visual object 939 for guiding the posture of the user 120. For example, the visual object 939 may be used to guide the posture of the user 120. For example, the visual object 939 may represent an example of the correct posture of the user 120 and an example of the incorrect posture of the user 120. For example, the external electronic device 130 may further display a visual object 936. For example, the visual object 936 may be described as a visual object for changing a setting for providing a notification based on the distance between the wearable device 100 and the external electronic device 110. For example, the external electronic device 130 may cause a change in the setting for the notification of the wearable device 100 based on receiving a user input for the visual object 936.

Referring to FIG. 9C, a state 950 may be described as a state in which a screen capable of changing the setting for the notification is displayed through the external electronic device 130. For example, the external electronic device 130 may display a visual object 952. For example, the visual object 952 may include a first visual object for changing a notification setting. For example, the first visual object may correspond to the visual object 750 and/or the visual object 760 of FIG. 7B. For example, the visual object 952 may include a second visual object for measuring a reference distance between the wearable device 100 and the external electronic device 110. For example, the second visual object may correspond to the visual object 712 of FIG. 7A. For example, the visual object 952 may include a third visual object that guides the posture of the user 120. For example, the third visual object may correspond to the visual object 822 and the visual object 824 of FIG. 8B.

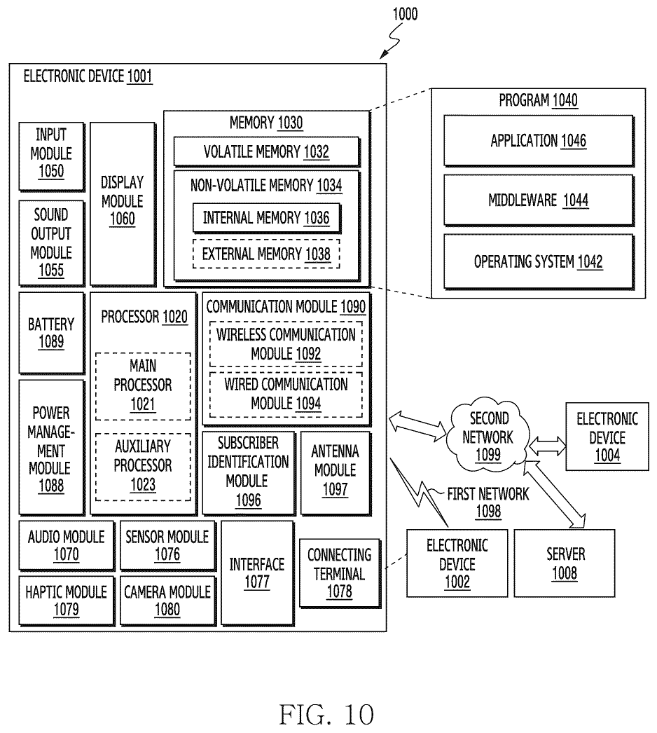

FIG. 10 is a block diagram of an electronic device in a network environment according to various embodiments.

Referring to FIG. 10, the electronic device 1001 in the network environment 1000 may communicate with an electronic device 1002 via a first network 1098 (e.g., a short-range wireless communication network), or at least one of an electronic device 1004 or a server 1008 via a second network 1099 (e.g., a long-range wireless communication network). According to an embodiment, the electronic device 1001 may communicate with the electronic device 1004 via the server 1008. According to an embodiment, the electronic device 1001 may include a processor 1020, memory 1030, an input module 1050, a sound output module 1055, a display module 1060, an audio module 1070, a sensor module 1076, an interface 1077, a connecting terminal 1078, a haptic module 1079, a camera module 1080, a power management module 1088, a battery 1089, a communication module 1090, a subscriber identification module(SIM) 1096, or an antenna module 1097. In some embodiments, at least one of the components (e.g., the connecting terminal 1078) may be omitted from the electronic device 1001, or one or more other components may be added in the electronic device 1001. In some embodiments, some of the components (e.g., the sensor module 1076, the camera module 1080, or the antenna module 1097) may be implemented as a single component (e.g., the display module 1060).

The processor 1020 may execute, for example, software (e.g., a program 1040) to control at least one other component (e.g., a hardware or software component) of the electronic device 1001 coupled with the processor 1020, and may perform various data processing or computation. According to an embodiment, as at least part of the data processing or computation, the processor 1020 may store a command or data received from another component (e.g., the sensor module 1076 or the communication module 1090) in volatile memory 1032, process the command or the data stored in the volatile memory 1032, and store resulting data in non-volatile memory 1034. According to an embodiment, the processor 1020 may include a main processor 1021 (e.g., a central processing unit (CPU) or an application processor (AP)), or an auxiliary processor 1023 (e.g., a graphics processing unit (GPU), a neural processing unit (NPU), an image signal processor (ISP), a sensor hub processor, or a communication processor (CP)) that is operable independently from, or in conjunction with, the main processor 1021. For example, when the electronic device 1001 includes the main processor 1021 and the auxiliary processor 1023, the auxiliary processor 1023 may be adapted to consume less power than the main processor 1021, or to be specific to a specified function. The auxiliary processor 1023 may be implemented as separate from, or as part of the main processor 1021.

The auxiliary processor 1023 may control at least some of functions or states related to at least one component (e.g., the display module 1060, the sensor module 1076, or the communication module 1090) among the components of the electronic device 1001, instead of the main processor 1021 while the main processor 1021 is in an inactive (e.g., sleep) state, or together with the main processor 1021 while the main processor 1021 is in an active state (e.g., executing an application). According to an embodiment, the auxiliary processor 1023 (e.g., an image signal processor or a communication processor) may be implemented as part of another component (e.g., the camera module 1080 or the communication module 1090) functionally related to the auxiliary processor 1023. According to an embodiment, the auxiliary processor 1023 (e.g., the neural processing unit) may include a hardware structure specified for artificial intelligence model processing. An artificial intelligence model may be generated by machine learning. Such learning may be performed, e.g., by the electronic device 1001 where the artificial intelligence is performed or via a separate server (e.g., the server 1008). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 1030 may store various data used by at least one component (e.g., the processor 1020 or the sensor module 1076) of the electronic device 1001. The various data may include, for example, software (e.g., the program 1040) and input data or output data for a command related thereto. The memory 1030 may include the volatile memory 1032 or the non-volatile memory 1034.

The program 1040 may be stored in the memory 1030 as software, and may include, for example, an operating system (OS) 1042, middleware 1044, or an application 1046.

The input module 1050 may receive a command or data to be used by another component (e.g., the processor 1020) of the electronic device 1001, from the outside (e.g., a user) of the electronic device 1001. The input module 1050 may include, for example, a microphone, a mouse, a keyboard, a key (e.g., a button), or a digital pen (e.g., a stylus pen).

The sound output module 1055 may output sound signals to the outside of the electronic device 1001. The sound output module 1055 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 1060 may visually provide information to the outside (e.g., a user) of the electronic device 1001. The display module 1060 may include, for example, a display, a hologram device, or a projector and control circuitry to control a corresponding one of the display, hologram device, and projector. According to an embodiment, the display module 1060 may include a touch sensor adapted to detect a touch, or a pressure sensor adapted to measure the intensity of force incurred by the touch.

The audio module 1070 may convert a sound into an electrical signal and vice versa. According to an embodiment, the audio module 1070 may obtain the sound via the input module 1050, or output the sound via the sound output module 1055 or a headphone of an external electronic device (e.g., an electronic device 1002) directly (e.g., wiredly) or wirelessly coupled with the electronic device 1001.

The sensor module 1076 may detect an operational state (e.g., power or temperature) of the electronic device 1001 or an environmental state (e.g., a state of a user) external to the electronic device 1001, and then generate an electrical signal or data value corresponding to the detected state. According to an embodiment, the sensor module 1076 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 1077 may support one or more specified protocols to be used for the electronic device 1001 to be coupled with the external electronic device (e.g., the electronic device 1002) directly (e.g., wiredly) or wirelessly. According to an embodiment, the interface 1077 may include, for example, a high definition multimedia interface (HDMI), a universal serial bus (USB) interface, a secure digital (SD) card interface, or an audio interface.

A connecting terminal 1078 may include a connector via which the electronic device 1001 may be physically connected with the external electronic device (e.g., the electronic device 1002). According to an embodiment, the connecting terminal 1078 may include, for example, an HDMI connector, a USB connector, an SD card connector, or an audio connector (e.g., a headphone connector).

The haptic module 1079 may convert an electrical signal into a mechanical stimulus (e.g., a vibration or a movement) or electrical stimulus which may be recognized by a user via his tactile sensation or kinesthetic sensation. According to an embodiment, the haptic module 1079 may include, for example, a motor, a piezoelectric element, or an electric stimulator.

The camera module 1080 may capture a still image or moving images. According to an embodiment, the camera module 1080 may include one or more lenses, image sensors, image signal processors, or flashes.

The power management module 1088 may manage power supplied to the electronic device 1001. According to an embodiment, the power management module 1088 may be implemented as at least part of, for example, a power management integrated circuit (PMIC).

The battery 1089 may supply power to at least one component of the electronic device 1001. According to an embodiment, the battery 1089 may include, for example, a primary cell which is not rechargeable, a secondary cell which is rechargeable, or a fuel cell.

The communication module 1090 may support establishing a direct (e.g., wired) communication channel or a wireless communication channel between the electronic device 1001 and the external electronic device (e.g., the electronic device 1002, the electronic device 1004, or the server 1008) and performing communication via the established communication channel. The communication module 1090 may include one or more communication processors that are operable independently from the processor 1020 (e.g., the application processor (AP)) and supports a direct (e.g., wired) communication or a wireless communication. According to an embodiment, the communication module 1090 may include a wireless communication module 1092 (e.g., a cellular communication module, a short-range wireless communication module, or a global navigation satellite system (GNSS) communication module) or a wired communication module 1094 (e.g., a local area network (LAN) communication module or a power line communication (PLC) module). A corresponding one of these communication modules may communicate with the external electronic device via the first network 1098 (e.g., a short-range communication network, such as Bluetooth™, wireless-fidelity (Wi-Fi) direct, or infrared data association (IrDA)) or the second network 1099 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 1092 may identify and authenticate the electronic device 1001 in a communication network, such as the first network 1098 or the second network 1099, using subscriber information (e.g., international mobile subscriber identity (IMSI)) stored in the subscriber identification module 1096.

The wireless communication module 1092 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 1092 may support a high-frequency band (e.g., the mmWave band) to achieve, e.g., a high data transmission rate. The wireless communication module 1092 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 1092 may support various requirements specified in the electronic device 1001, an external electronic device (e.g., the electronic device 1004), or a network system (e.g., the second network 1099). According to an embodiment, the wireless communication module 1092 may support a peak data rate (e.g., 20 Gbps or more) for implementing eMBB, loss coverage (e.g., 1064 dB or less) for implementing mMTC, or U-plane latency (e.g., 0.5 ms or less for each of downlink (DL) and uplink (UL), or a round trip of 10 ms or less) for implementing URLLC.

The antenna module 1097 may transmit or receive a signal or power to or from the outside (e.g., the external electronic device) of the electronic device 1001. According to an embodiment, the antenna module 1097 may include an antenna including a radiating element composed of a conductive material or a conductive pattern formed in or on a substrate (e.g., a printed circuit board (PCB)). According to an embodiment, the antenna module 1097 may include a plurality of antennas (e.g., array antennas). In such a case, at least one antenna appropriate for a communication scheme used in the communication network, such as the first network 1098 or the second network 1099, may be selected, for example, by the communication module 1090 (e.g., the wireless communication module 1092) from the plurality of antennas. The signal or the power may then be transmitted or received between the communication module 1090 and the external electronic device via the selected at least one antenna. According to an embodiment, another component (e.g., a radio frequency integrated circuit (RFIC)) other than the radiating element may be additionally formed as part of the antenna module 1097.

According to various embodiments, the antenna module 1097 may form a mmWave antenna module. According to an embodiment, the mmWave antenna module may include a printed circuit board, an RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 1001 and the external electronic device 1004 via the server 1008 coupled with the second network 1099. Each of the electronic devices 1002 or 1004 may be a device of a same type as, or a different type, from the electronic device 1001. According to an embodiment, all or some of operations to be executed at the electronic device 1001 may be executed at one or more of the external electronic devices 1002, 1004, or 1008. For example, if the electronic device 1001 should perform a function or a service automatically, or in response to a request from a user or another device, the electronic device 1001, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 1001. The electronic device 1001 may provide the outcome, with or without further processing of the outcome, as at least part of a reply to the request. To that end, a cloud computing, distributed computing, mobile edge computing (MEC), or client-server computing technology may be used, for example. The electronic device 1001 may provide ultra low-latency services using, e.g., distributed computing or mobile edge computing. In another embodiment, the external electronic device 1004 may include an internet-of-things (IoT) device. The server 1008 may be an intelligent server using machine learning and/or a neural network. According to an embodiment, the external electronic device 1004 or the server 1008 may be included in the second network 1099. The electronic device 1001 may be applied to intelligent services (e.g., smart home, smart city, smart car, or healthcare) based on 5G communication technology or IoT-related technology.

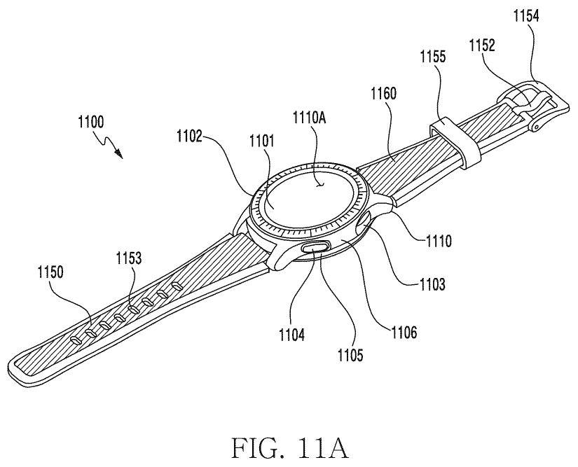

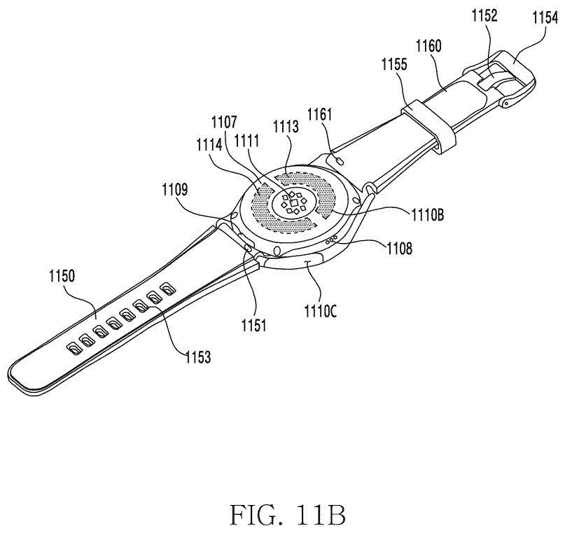

FIGS. 11A and 11B illustrate perspective views of an exemplary electronic device according to an embodiment.

Referring to FIGS. 11A and 11B, an electronic device 1100 (e.g., the electronic device 1001 of FIG. 10) according to an embodiment may include a housing 1110 including a first surface (or a front surface) 1110A, a second surface (or a rear surface) 1110B, and a side surface 1110C surrounding a space between the first surface 1110A and the second surface 1110B, and fastening members 1150 and 1160 connected to at least a portion of the housing 1110 and configured to detachably fasten the electronic device 1100 to a part (e.g., a wrist or an ankle) of a user's body. In another embodiment (not illustrated), the housing may refer to a structure forming a portion of the first surface 1110A, the second surface 1110B, and the side surface 1110C of FIGS. 11A and 11B. According to an embodiment, the first surface 1110A may be formed by a front plate 1101 transparent (e.g., a glass plate or a polymer plate including various coating layers) that is at least partially substantially. The second surface 1110B may be formed by a rear plate 1107 that is substantially opaque. The rear plate 1107 may be formed by, for example, coated or colored glass, ceramic, polymer, metal (e.g., aluminum, stainless steel (STS), or magnesium), or a combination of at least two of the materials. The side surface 1110C may be coupled to the front plate 1101 and the rear plate 1107 and may be formed by a side bezel structure (or “a side member”) 1106 including metal and/or polymer. In some embodiments, the rear plate 1107 and the side bezel structure 1106 may be integrally formed and include the same material (e.g., a metal material such as aluminum). The fastening members 1150 and 1160 may be formed of various materials and shapes. Integral and a plurality of unit links may be formed to be movable to each other by woven fabric, leather, rubber, urethane, metal, ceramic, or a combination of at least two of the materials.

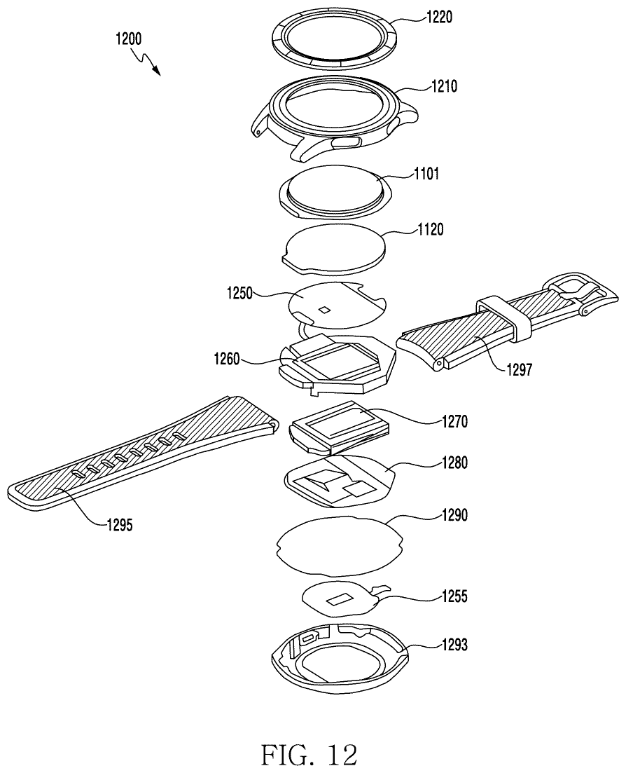

According to an embodiment, the electronic device 1100 may include at least one or more of a display 1120 (refer to FIG. 12), audio modules 1105 and 1108, a sensor module 1111, key input devices 1102, 1103, and 1104, and a connector hole 1109. In some embodiments, the electronic device 1100 may omit at least one of components (e.g., the key input devices 1102, 1103, and 1104, the connector hole 1109, or the sensor module 1111) or may additionally include another component.

The display 1120 may be visually exposed, for example, through a significant portion of the front plate 1101. A shape of the display 1120 may be a shape corresponding to a shape of the front plate 1101 and may be various shapes such as a circle, an oval, or a polygon. The display 1120 may be coupled to or disposed adjacent to touch detection circuitry, a pressure sensor capable of measuring an intensity (pressure) of a touch, and/or a fingerprint sensor.

The audio modules 1105 and 1108 may include a microphone hole 1105 and a speaker hole 1108. In the microphone hole 1105A, a microphone for obtaining an external sound may be disposed in the inside, and in some embodiments, a plurality of microphones may be disposed to detect a direction of a sound. The speaker hole 1108 may be used as an external speaker and a call receiver. In some embodiments, the speaker hole 1108 and the microphone hole 1105 may be implemented as one hole, or a speaker may be included without the speaker hole 1108 (e.g., a piezo speaker).

The sensor module 1111 may generate an electrical signal or a data value corresponding to an internal operating state or an external environmental state of the electronic device 1100. The sensor module 1111 may include, for example, a biometric sensor module 1111 (e.g., an HRM sensor) disposed on the second surface 1110B of the housing 1110. The electronic device 1100 may further include at least one of a sensor module not illustrated, for example, a gesture sensor, a gyro sensor, a barometric sensor, a magnetic sensor, an acceleration sensor, a grip sensor, a color sensor, an infrared (IR) sensor, a biometric sensor, a temperature sensor, a humidity sensor, or an illuminance sensor.

The sensor module 1111 may include electrode regions 1113 and 1114 forming a portion of a surface of the electronic device 1100 and biometric signal detection circuitry (not illustrated) electrically connected to the electrode regions 1113 and 1114. For example, the electrode regions 1113 and 1114 may include the first electrode region 1113 and the second electrode region 1114 disposed on the second surface 1110B of the housing 1110. The sensor module 1111 may be configured such that the electrode regions 1113 and 1114 obtain an electrical signal from a part of the user's body, and the biometric signal detection circuitry detects biometric information of the user based on the electrical signal.

The key input devices 1102, 1103, and 1104 may include the wheel key 1102 disposed on the first surface 1110A of the housing 1110 and rotatable in at least one direction, and/or the side key buttons 1103 and 1104 disposed on the side surface 1110C of the housing 1110. The wheel key may have a shape corresponding to the shape of the front plate 1101. In another embodiment, the electronic device 1100 may not include a portion or all of the key input devices 1102, 1103, and 1104 mentioned above, and the key input devices 1102, 1103, and 1104 that are not included may be implemented in another form such as a soft key and the like on the display 1120. The connector hole 1109 may accommodate a connector (e.g., a USB connector) for transmitting and receiving power and/or data with an external electronic device and may include another connector hole (not illustrated) that may accommodate a connector for transmitting and receiving an audio signal with the external electronic device. The electronic device 1100 may further include, for example, a connector cover (not illustrated) covering at least a portion of the connector hole 1109 and blocking an inflow of an external foreign substance into the connector hole.

The fastening members 1150 and 1160 may be detachably fastened to at least a portion of the housing 1110 using locking members 1151 and 1161. The fastening members 1150 and 1160 may include one or more of a fixing member 1152, a fixing member fastening hole 1153, a band guide member 1154, and a band fixing ring 1155.

The fixing member 1152 may be configured to fix the housing 1110 and the fastening members 1150 and 1160 to a part (e.g., the wrist or the ankle) of the user's body. The fixing member fastening hole 1153 may fix the housing 1110 and the fastening members 1150 and 1160 to the part of the user's body in response to the fixing member 1152. The band guide member 1154 may allow the fastening members 1150 and 1160 to be fastened in close contact with the part of the user's body, by being configured to limit a movement range of the fixing member 1152 when the fixing member 1152 is fastened to the fixing member fastening hole 1153. The band fixing ring 1155 may limit a movement range of the fastening members 1150 and 1160 in a state in which the fixing member 1152 and the fixing member fastening hole 1153 are fastened.

FIG. 12 illustrates an exploded perspective view of an exemplary electronic device according to an embodiment.

Referring to FIG. 12, an electronic device 1200 (e.g., the electronic device 1001 of FIG. 10 or the electronic device 1100 of FIGS. 11A to 11B) may include a side bezel structure 1210, a wheel key 1220 (e.g., the wheel key 1102 of FIG. 11A), a front plate 1101, a display 1120, a first antenna 1250, a second antenna 1255, a support member 1260 (e.g., a bracket), a battery 1270, a printed circuit board 1280, a sealing member 1290, a rear plate 1293 (e.g., the rear plate 1107 of FIG. 11B) and fastening members 1295 and 1297 (e.g., the fastening members 1150 and 1160 of FIG. 11B). At least one of components of the electronic device 1200 may be the same as or similar to at least one of the components of the electronic device 1100 of FIG. 10 or FIGS. 11A to 11B, and a redundant description will be omitted below. The support member 1260 may be disposed inside the electronic device 1200 and connected to the side bezel structure 1210, or may be integrally formed with the side bezel structure 1210. The support member 1260 may be formed of, for example, a metal material and/or a non-metal (e.g., polymer) material. In the support member 1260, the display 1120 may be coupled to a surface and the printed circuit board 1280 may be coupled to another surface. A processor, memory, and/or an interface may be mounted on the printed circuit board 1280. The processor may include, for example, one or more of a central processing unit, a graphic processing unit (GPU), an application processor, a sensor processor, or a communication processor.

The memory may include, for example, volatile memory or non-volatile memory. The interface may include, for example, a high definition multimedia interface (HDMI), a universal serial bus (USB) interface, an SD card interface, and/or an audio interface. For example, the interface may electrically or physically connect the electronic device 1200 to an external electronic device and may include a USB connector, an SD card/MMC connector, or an audio connector.

The battery 1270 is a device for supplying power to at least one component of the electronic device 1200, and may include, for example, a non-rechargeable primary battery, a rechargeable secondary battery, or a fuel cell. At least a portion of the battery 1270 may be disposed on substantially the same plane as, for example, the printed circuit board 1280. The battery 1270 may be integrally disposed inside the electronic device 1100 or may be detachably disposed from the electronic device 1100.

The first antenna 1250 may be disposed between the display 1120 and the support member 1260. The first antenna 1250 may include, for example, a near field communication (NFC) antenna, a wireless charging antenna, and/or a magnetic secure transmission (MST) antenna. The first antenna 1250 may, for example, perform short-range communication with an external device, wirelessly transmit and receive power required for charging, and transmit a magnetic-based signal including a short-range communication signal or payment data. In another embodiment, an antenna structure may be formed by a portion or a combination of the side bezel structure 1210 and/or the support member 1260.

The second antenna 1255 may be disposed between the printed circuit board 1280 and the rear plate 1293. The second antenna 1255 may include, for example, a near field communication (NFC) antenna, a wireless charging antenna, and/or a magnetic secure transmission (MST) antenna. The second antenna 1255 may, for example, perform short-range communication with an external device, wirelessly transmit and receive power required for charging, and transmit a magnetic-based signal including a short-range communication signal or payment data. In another embodiment, an antenna structure may be formed by a portion or a combination of the side bezel structure 1210 and/or the rear plate 1293.

The sealing member 1290 may be positioned between the side bezel structure 1210 and the rear plate 1293. The sealing member 1290 may be configured to block moisture and a foreign substance flowing into a space surrounded by the side bezel structure 1210 and the rear plate 1293 from the outside.

The wearable device described above may correspond to the electronic device 1001 of FIG. 10, the electronic device 1100 of FIGS. 11A and 11B, and/or the electronic device 1200 of FIG. 12.

The technical problems to be achieved in the present disclosure are not limited to those described above, and other technical problems not mentioned herein will be clearly understood by those having ordinary knowledge in the art to which the present disclosure belongs.

As described above, a wearable device (e.g., the wearable device 100) may comprise memory (e.g., the memory 206) comprising one or more storage media storing instructions. The wearable device may comprise a display (e.g., the display 208). The wearable device may comprise communication circuitry (e.g., the communication circuitry 205). The wearable device may comprise at least one processor (e.g., the at least one processor 207) comprising processing circuitry. The instructions, when executed by the at least one processor individually or collectively, may cause the wearable device to, while being connected to an external electronic device 110 through the communication circuitry, obtain information associated with a distance between the wearable device 100 and the external electronic device 110. The instructions, when executed by the at least one processor individually or collectively, may cause the wearable device to, by using the information, identify whether the distance is included in a range that is set according to a reference distance between the wearable device 100 and the external electronic device 110. The reference distance may be obtained before the information is obtained. The instructions, when executed by the at least one processor individually or collectively, may cause the wearable device to, based on obtaining the distance outside the range, identify time during which the distance has been maintained outside the range. The instructions, when executed by the at least one processor individually or collectively, may cause the wearable device to, based on obtaining the distance outside the range, based on identifying the time greater than threshold time, display, through the display 208, a first user interface (UI) object 925 indicating that a pose of a user of the wearable device is corresponding to a first pose.

According to an embodiment, the range may be set to define the first pose of the user wearing the wearable device and the external electronic device. The instructions, when executed by the at least one processor individually or collectively, may cause the wearable device to, based on identifying that the distance is included in the range, identify other time during which the distance has been maintained in the range. The acceleration sensor may be periodically enabled according to a first period. The instructions, when executed by the at least one processor individually or collectively, may cause the wearable device to, based on identifying that the distance is included in the range, based on identifying the other time greater than other threshold time, display, through the display 208, a second UI object indicating that a pose of the user 120 corresponds to the second pose.

According to an embodiment, the instructions, when executed by the at least one processor individually or collectively, may cause the wearable device to, by using the information, identify acceleration of the external electronic device at least based on an acceleration sensor included in the external electronic device. The acceleration sensor may be periodically enabled according to a first period. The instructions, when executed by the at least one processor individually or collectively, may cause the wearable device to, based on identifying the acceleration lower than threshold acceleration, transmit, through the communication circuitry to the external electronic device, a signal instructing to change a period of the acceleration sensor to a second period longer than the first period. The instructions, when executed by the at least one processor individually or collectively, may cause the wearable device to, based on identifying the acceleration higher than the threshold acceleration, transmit, through the communication circuitry to the external electronic device, another signal instructing to maintain the period of the acceleration sensor as the first period.

According to an embodiment, the wearable device may further comprise an acceleration sensor. The instructions, when executed by the at least one processor individually or collectively, may cause the wearable device to identify, through the acceleration sensor, acceleration of the wearable device. The instructions, when executed by the at least one processor individually or collectively, may cause the wearable device to, based on determination that the acceleration is higher than threshold acceleration, obtain, in a first period, the information. The instructions, when executed by the at least one processor individually or collectively, may cause the wearable device to, based on determination that the acceleration is lower than the threshold acceleration, obtain, in a second period longer than the first period, the information.

According to an embodiment, the wearable device may further comprise a rechargeable battery. The instructions, when executed by the at least one processor individually or collectively, may cause the wearable device to, based on determination that a state of charge (SoC) of the rechargeable battery is higher than a threshold SoC, obtain, in a first period, the information. The instructions, when executed by the at least one processor individually or collectively, may cause the wearable device to, based on determination that the SoC of the rechargeable battery is lower than the threshold SoC, obtain, in a second period longer than the first period, the information.

According to an embodiment, the instructions, when executed by the at least one processor individually or collectively, may cause the wearable device to, while displaying the first UI object, obtain other information associated with the distance between the wearable device and the external electronic device. The instructions, when executed by the at least one processor individually or collectively, may cause the wearable device to, based on identifying a distance indicated by the other information being included in the range, display, through the display, a third UI object indicating that a pose of the user corresponds to the second pose.

According to an embodiment, the information may further include information associated with a tilt of the external electronic device. The instructions, when executed by the at least one processor individually or collectively, may cause the wearable device to, by using the information, identify the tilt of the external electronic device. The instructions, when executed by the at least one processor individually or collectively, may cause the wearable device to, based on identifying the tilt, identify whether the tilt is included in another range set according to a reference tilt of the external electronic device that was obtained before obtaining the information. The instructions, when executed by the at least one processor individually or collectively, may cause the wearable device to, based on obtaining the tilt included in the other range for defining the pose of the user, identify the time during which the tilt has been maintained in the other range.

According to an embodiment, the instructions, when executed by the at least one processor individually or collectively, may cause the wearable device to, before obtaining the information, when executing a software application for monitoring a body pose, display, through the display, a screen indicating a reference pose provided from the software application. The instructions, when executed by the at least one processor individually or collectively, may cause the wearable device to, based on receiving a user input with respect to the screen, obtain the reference distance between the wearable device and the external electronic device.

According to an embodiment, the wearable device may further comprise a speaker and an actuator. The instructions, when executed by the at least one processor individually or collectively, may cause the wearable device to, when executing a software application for monitoring a body pose, display, through the display, a screen to set a manner of a notification. The instructions, when executed by the at least one processor individually or collectively, may cause the wearable device to, based on receiving a user input with respect to the screen, when identifying that the time is greater than the threshold time, perform at least one among displaying the first UI object through the display, outputting an audio signal corresponding to the notification through the speaker, and controlling the actuator for providing a vibration notification.

According to an embodiment, the instructions, when executed by the at least one processor individually or collectively, may cause the wearable device to, when executing a software application for monitoring a body pose, display, through the display, a screen to set the threshold time. The instructions, when executed by the at least one processor individually or collectively, may cause the wearable device to, based on receiving a user input with respect to the screen, determine the threshold time.

According to an embodiment, the wearable device may further comprise an actuator. The instructions, when executed by the at least one processor individually or collectively, may cause the wearable device to, based on obtaining the distance outside the range, identify the time during which the distance has been maintained outside the range. The instructions, when executed by the at least one processor individually or collectively, may cause the wearable device to, based on obtaining the distance outside the range, based on identifying the time greater than the threshold time, control the actuator to provide a vibration notification at a first intensity. The instructions, when executed by the at least one processor individually or collectively, may cause the wearable device to, based on obtaining the distance outside the range, based on identifying other time greater than the time, control the actuator to provide the vibration notification at a second intensity.

According to an embodiment, the wearable device may further comprise a time of flight (ToF) sensor. The information may be obtained through the ToF sensor.

According to an embodiment, the range may be set based on a first value for a first ratio of the reference distance and a second value for a second ratio of the reference distance.

According to an embodiment, the information may be obtained based on a received signal strength indicator (RSSI) using the communication circuitry.

As described above, a method executed in a wearable device (e.g., the wearable device 100) with a display (e.g., the display 208) and communication circuitry (e.g., the communication circuitry 205) may comprise, while being connected to an external electronic device 110 through the communication circuitry, obtaining information associated with a distance between the wearable device 100 and the external electronic device 110. The method may comprise, by using the information, identifying whether the distance is included in a range that is set according to a reference distance between the wearable device 100 and the external electronic device 110. The reference distance may be obtained before the information is obtained. The method may comprise, based on obtaining the distance outside the range, identifying time during which the distance has been maintained outside the range. The method may comprise, based on obtaining the distance outside the range, based on identifying the time greater than threshold time, displaying, through the display 208, a first user interface (UI) object 925 indicating that a pose of a user of the wearable device is corresponding to a first pose.

According to an embodiment, the range may be set to define the first pose of the user wearing the wearable device and the external electronic device. The method may comprise, based on identifying that the distance is included in the range, identifying other time during which the distance has been maintained in the range. The method may comprise, based on identifying that the distance is included in the range, based on identifying the other time greater than other threshold time, displaying, through the display 208, a second UI object indicating that a pose of the user 120 corresponds to the second pose.

According to an embodiment, the method may comprise, by using the information, identifying acceleration of the external electronic device at least based on an acceleration sensor included in the external electronic device. The acceleration sensor may be periodically enabled according to a first period. The method may comprise, based on identifying the acceleration lower than threshold acceleration, transmitting, through the communication circuitry to the external electronic device, a signal instructing to change a period of the acceleration sensor to a second period longer than the first period. The method may comprise, based on identifying the acceleration higher than the threshold acceleration, transmitting, through the communication circuitry to the external electronic device, another signal instructing to maintain the period of the acceleration sensor as the first period.

According to an embodiment, the wearable device may further comprise an acceleration sensor. The method may comprise identifying, through the acceleration sensor, acceleration of the wearable device. The method may comprise, based on determination that the acceleration is higher than threshold acceleration, obtaining, in a first period, the information. The method may comprise, based on determination that the acceleration is lower than the threshold acceleration, obtaining, in a second period longer than the first period, the information.

According to an embodiment, the wearable device may further comprise a rechargeable battery. The method may comprise, based on determination that a state of charge (SoC) of the rechargeable battery is higher than a threshold SoC, obtaining, in a first period, the information. The method may comprise, based on determination that the SoC of the rechargeable battery is lower than the threshold SoC, obtaining, in a second period longer than the first period, the information.

According to an embodiment, the method may comprise, while displaying the first UI object, obtaining other information associated with the distance between the wearable device and the external electronic device. The method may comprise, based on identifying a distance indicated by the other information being included in the range, displaying, through the display, a third UI object indicating that a pose of the user corresponds to the second pose.

According to an embodiment, the information may further include information associated with a tilt of the external electronic device. The method may comprise, by using the information, identifying the tilt of the external electronic device. The method may comprise, based on identifying the tilt, identifying whether the tilt is included in another range set according to a reference tilt of the external electronic device that was obtained before obtaining the information. The method may comprise, based on obtaining the tilt included in the other range for defining the pose of the user, identifying the time during which the tilt has been maintained in the other range.

According to an embodiment, the method may comprise, before obtaining the information, when executing a software application for monitoring a body pose, displaying, through the display, a screen indicating a reference pose provided from the software application. The method may comprise, based on receiving a user input with respect to the screen, obtaining the reference distance between the wearable device and the external electronic device.

According to an embodiment, the wearable device may further comprise a speaker and an actuator. The method may comprise, when executing a software application for monitoring a body pose, displaying, through the display, a screen to set a manner of a notification. The method may comprise, based on receiving a user input with respect to the screen, when identifying that the time is greater than the threshold time, performing at least one among displaying the first UI object through the display, outputting an audio signal corresponding to the notification through the speaker, and controlling the actuator for providing a vibration notification.

According to an embodiment, the method may comprise, when executing a software application for monitoring a body pose, displaying, through the display, a screen to set the threshold time. The method may comprise, based on receiving a user input with respect to the screen, determining the threshold time.

According to an embodiment, the wearable device may further comprise an actuator. The method may comprise, based on obtaining the distance outside the range, identifying the time during which the distance has been maintained outside the range. The method may comprise, based on obtaining the distance outside the range, based on identifying the time greater than the threshold time, controlling the actuator to provide a vibration notification at a first intensity. The method may comprise, based on obtaining the distance outside the range, based on identifying other time greater than the time, controlling the actuator to provide the vibration notification at a second intensity.

According to an embodiment, the wearable device may further comprise a time of flight (ToF) sensor. The information may be obtained through the ToF sensor.

According to an embodiment, the range may be set based on a first value for a first ratio of the reference distance and a second value for a second ratio of the reference distance.

According to an embodiment, the information may be obtained based on a received signal strength indicator (RSSI) using the communication circuitry.

As described above, in a non-transitory computer readable storage medium storing one or more programs, the one or more programs may comprise instructions to, when executed by a wearable device (e.g., the wearable device 100) with (e.g., the display 208) and communication circuitry (e.g., the communication circuitry 205), cause the wearable device to, while being connected to an external electronic device 110 through the communication circuitry, obtain information associated with a distance between the wearable device 100 and the external electronic device 110. The one or more programs may comprise instructions to, when executed by the wearable device, cause the wearable device to, by using the information, identify whether the distance is included in a range that is set according to a reference distance between the wearable device 100 and the external electronic device 110. The reference distance may be obtained before the information is obtained. The one or more programs may comprise instructions to, when executed by the wearable device, cause the wearable device to, based on obtaining the distance outside the range, identify time during which the distance has been maintained outside the range. The one or more programs may comprise instructions to, when executed by the wearable device, cause the wearable device to, based on obtaining the distance outside the range, based on identifying the time greater than threshold time, display, through the display 208, a first user interface (UI) object 925 indicating that a pose of a user of the wearable device is corresponding to a first pose.

According to an embodiment, the range may be set to define the first pose of the user wearing the wearable device and the external electronic device. The one or more programs may comprise instructions to, when executed by the wearable device, cause the wearable device to, based on identifying that the distance is included in the range, identify other time during which the distance has been maintained in the range. The one or more programs may comprise instructions to, when executed by the wearable device, cause the wearable device to, based on identifying that the distance is included in the range, based on identifying the other time greater than other threshold time, display, through the display 208, a second UI object indicating that a pose of the user 120 corresponds to the second pose.

According to an embodiment, the one or more programs may comprise instructions to, when executed by the wearable device, cause the wearable device to, by using the information, identify acceleration of the external electronic device at least based on an acceleration sensor included in the external electronic device. The acceleration sensor may be periodically enabled according to a first period. The one or more programs may comprise instructions to, when executed by the wearable device, cause the wearable device to, based on identifying the acceleration lower than threshold acceleration, transmit, through the communication circuitry to the external electronic device, a signal instructing to change a period of the acceleration sensor to a second period longer than the first period. The one or more programs may comprise instructions to, when executed by the wearable device, cause the wearable device to, based on identifying the acceleration higher than the threshold acceleration, transmit, through the communication circuitry to the external electronic device, another signal instructing to maintain the period of the acceleration sensor as the first period.

According to an embodiment, the wearable device may further comprise an acceleration sensor. The one or more programs may comprise instructions to, when executed by the wearable device, cause the wearable device to identify, through the acceleration sensor, acceleration of the wearable device. The one or more programs may comprise instructions to, when executed by the wearable device, cause the wearable device to, based on determination that the acceleration is higher than threshold acceleration, obtain, in a first period, the information. The one or more programs may comprise instructions to, when executed by the wearable device, cause the wearable device to, based on determination that the acceleration is lower than the threshold acceleration, obtain, in a second period longer than the first period, the information.

According to an embodiment, the wearable device may further comprise a rechargeable battery. The one or more programs may comprise instructions to, when executed by the wearable device, cause the wearable device to, based on determination that a state of charge (SoC) of the rechargeable battery is higher than a threshold SoC, obtain, in a first period, the information. The one or more programs may comprise instructions to, when executed by the wearable device, cause the wearable device to, based on determination that the SoC of the rechargeable battery is lower than the threshold SoC, obtain, in a second period longer than the first period, the information.

According to an embodiment, the one or more programs may comprise instructions to, when executed by the wearable device, cause the wearable device to, while displaying the first UI object, obtain other information associated with the distance between the wearable device and the external electronic device. The one or more programs may comprise instructions to, when executed by the wearable device, cause the wearable device to, based on identifying a distance indicated by the other information being included in the range, display, through the display, a third UI object indicating that a pose of the user corresponds to the second pose.

According to an embodiment, the information may further include information associated with a tilt of the external electronic device. The one or more programs may comprise instructions to, when executed by the wearable device, cause the wearable device to, by using the information, identify the tilt of the external electronic device. The one or more programs may comprise instructions to, when executed by the wearable device, cause the wearable device to, based on identifying the tilt, identify whether the tilt is included in another range set according to a reference tilt of the external electronic device that was obtained before obtaining the information. The one or more programs may comprise instructions to, when executed by the wearable device, cause the wearable device to, based on obtaining the tilt included in the other range for defining the pose of the user, identify the time during which the tilt has been maintained in the other range.

According to an embodiment, the one or more programs may comprise instructions to, when executed by the wearable device, cause the wearable device to, before obtaining the information, when executing a software application for monitoring a body pose, display, through the display, a screen indicating a reference pose provided from the software application. The one or more programs may comprise instructions to, when executed by the wearable device, cause the wearable device to, based on receiving a user input with respect to the screen, obtain the reference distance between the wearable device and the external electronic device.

According to an embodiment, the wearable device may further comprise a speaker and an actuator. The one or more programs may comprise instructions to, when executed by the wearable device, cause the wearable device to, when executing a software application for monitoring a body pose, display, through the display, a screen to set a manner of a notification. The one or more programs may comprise instructions to, when executed by the wearable device, cause the wearable device to, based on receiving a user input with respect to the screen, when identifying that the time is greater than the threshold time, perform at least one among displaying the first UI object through the display, outputting an audio signal corresponding to the notification through the speaker, and controlling the actuator for providing a vibration notification.

According to an embodiment, the one or more programs may comprise instructions to, when executed by the wearable device, cause the wearable device to, when executing a software application for monitoring a body pose, display, through the display, a screen to set the threshold time. The one or more programs may comprise instructions to, when executed by the wearable device, cause the wearable device to, based on receiving a user input with respect to the screen, determine the threshold time.

According to an embodiment, the wearable device may further comprise an actuator. The one or more programs may comprise instructions to, when executed by the wearable device, cause the wearable device to, based on obtaining the distance outside the range, identify the time during which the distance has been maintained outside the range. The one or more programs may comprise instructions to, when executed by the wearable device, cause the wearable device to, based on obtaining the distance outside the range, based on identifying the time greater than the threshold time, control the actuator to provide a vibration notification at a first intensity. The one or more programs may comprise instructions to, when executed by the wearable device, cause the wearable device to, based on obtaining the distance outside the range, based on identifying other time greater than the time, control the actuator to provide the vibration notification at a second intensity.

According to an embodiment, the wearable device may further comprise a time of flight (ToF) sensor. The information may be obtained through the ToF sensor.

According to an embodiment, the range may be set based on a first value for a first ratio of the reference distance and a second value for a second ratio of the reference distance.

According to an embodiment, the information may be obtained based on a received signal strength indicator (RSSI) using the communication circuitry.

The effects that may be obtained from the present disclosure are not limited to those described above, and any other effects not mentioned herein will be clearly understood by those having ordinary knowledge in the art to which the present disclosure belongs.

The device described above may be implemented as a hardware component, a software component, and/or a combination of a hardware component and a software component. For example, the devices and components described in the embodiments may be implemented by using one or more general purpose computers or special purpose computers, such as a processor, controller, arithmetic logic unit (ALU), digital signal processor, microcomputer, field programmable gate array (FPGA), programmable logic unit (PLU), microprocessor, or any other device capable of executing and responding to instructions. The processing device may perform an operating system (OS) and one or more software applications executed on the operating system. In addition, the processing device may access, store, manipulate, process, and generate data in response to the execution of the software. For convenience of understanding, there is a case that one processing device is described as being used, but a person who has ordinary knowledge in the relevant technical field may see that the processing device may include a plurality of processing elements and/or a plurality of types of processing elements. For example, the processing device may include a plurality of processors or one processor and one controller. In addition, another processing configuration, such as a parallel processor, is also possible.

The software may include a computer program, code, instruction, or a combination of one or more thereof, and may configure the processing device to operate as desired or may command the processing device independently or collectively. The software and/or data may be embodied in any type of machine, component, physical device, computer storage medium, or device, to be interpreted by the processing device or to provide commands or data to the processing device. The software may be distributed on network-connected computer systems and stored or executed in a distributed manner. The software and data may be stored in one or more computer-readable recording medium.

The method according to the embodiment may be implemented in the form of a program command that may be performed through various computer means and recorded on a computer-readable medium. In this case, the medium may continuously store a program executable by the computer or may temporarily store the program for execution or download. In addition, the medium may be various recording means or storage means in the form of a single or a combination of several hardware, but is not limited to a medium directly connected to a certain computer system, and may exist distributed on the network. Examples of media may include a magnetic medium such as a hard disk, floppy disk, and magnetic tape, optical recording medium such as a CD-ROM and DVD, magneto-optical medium, such as a floptical disk, and those configured to store program instructions, including ROM, RAM, flash memory, and the like. In addition, examples of other media may include recording media or storage media managed by app stores that distribute applications, sites that supply or distribute various software, servers, and the like.

Although the embodiments have been described above with reference to limited examples and drawings, various modifications and variations may be made from the above description by those skilled in the art. For example, even if the described technologies are performed in a different order from the described method, and/or the components of the described system, structure, device, circuit, and the like are coupled or combined in a different form from the described method, or replaced or substituted by other components or equivalents, appropriate a result may be achieved.

Therefore, other implementations, other embodiments, and those equivalent to the scope of the claims are in the scope of the claims described later. According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

What is claimed is:

1. A wearable device comprising:
a display;
communication circuitry;
memory comprising one or more storage media storing instructions; and
at least one processor comprising processing circuitry,
wherein the instructions, when executed by the at least one processor individually or collectively, cause the wearable device to:
while being connected to an external electronic device through the communication circuitry, obtain information associated with a distance between the wearable device and the external electronic device,
by using the information, identify whether the distance is included in a range that is set according to a reference distance between the wearable device and the external electronic device,
based on obtaining the distance outside the range:
identify a time during which the distance has been maintained outside the range, and
based on identifying the time greater than a threshold time, display, through the display, a first user interface (UI) object indicating that a posture of a user of the wearable device is corresponding to a first posture,
wherein the reference distance is obtained before the information is obtained.

2. The wearable device of claim 1, wherein the range is set to define the first posture of the user wearing the wearable device and the external electronic device,
wherein the instructions, when executed by the at least one processor individually or collectively, cause the wearable device to:
based on identifying that the distance is included in the range:
identify another time during which the distance has been maintained in the range, and
based on identifying the another time greater than another threshold time, display, through the display, a second UI object indicating that the posture of the user corresponds to a second posture.

3. The wearable device of claim 1, wherein the instructions, when executed by the at least one processor individually or collectively, cause the wearable device to:
by using the information, identify acceleration of the external electronic device at least based on an acceleration sensor included in the external electronic device, the acceleration sensor being periodically enabled according to a first period;
based on identifying the acceleration lower than a threshold acceleration, transmit, through the communication circuitry to the external electronic device, a signal instructing to change a period of the acceleration sensor to a second period longer than the first period, and
based on identifying the acceleration higher than the threshold acceleration, transmit, through the communication circuitry to the external electronic device, another signal instructing to maintain the period of the acceleration sensor as the first period.

4. The wearable device of claim 1, wherein the wearable device further comprises an acceleration sensor,
wherein the instructions, when executed by the at least one processor individually or collectively, cause the wearable device to:
identify, through the acceleration sensor, acceleration of the wearable device,
based on a determination that the acceleration is higher than a threshold acceleration, obtain, in a first period, the information, and
based on a determination that the acceleration is lower than the threshold acceleration, obtain, in a second period longer than the first period, the information.

5. The wearable device of claim 1, wherein the wearable device further comprises a rechargeable battery,
wherein the instructions, when executed by the at least one processor individually or collectively, cause the wearable device to:
based on a determination that a state of charge (SoC) of the rechargeable battery is higher than a threshold SoC, obtain, in a first period, the information, and
based on a determination that the SoC of the rechargeable battery is lower than the threshold SoC, obtain, in a second period longer than the first period, the information.

6. The wearable device of claim 1, wherein the instructions, when executed by the at least one processor individually or collectively, cause the wearable device to:
while displaying the first UI object, obtain other information associated with the distance between the wearable device and the external electronic device; and
based on identifying another distance indicated by the other information being included in the range, display, through the display, a third UI object indicating that the posture of the user corresponds to the second posture.

7. The wearable device of claim 1, wherein the information further includes information associated with a tilt of the external electronic device,
wherein the instructions, when executed by the at least one processor individually or collectively, cause the wearable device to:
by using the information, identify the tilt of the external electronic device,
based on identifying the tilt, identify whether the tilt is included in another range set according to a reference tilt of the external electronic device that was obtained before obtaining the information, and
based on obtaining the tilt included in the another range for defining the posture of the user, identify the time during which the tilt has been maintained in the another range.

8. The wearable device of claim 1, wherein the instructions, when executed by the at least one processor individually or collectively, cause the wearable device to:
before obtaining the information, when executing a software application for monitoring a body posture, display, through the display, a screen indicating a reference posture provided from the software application, and
based on receiving a user input with respect to the screen, obtain the reference distance between the wearable device and the external electronic device.

9. The wearable device of claim 1, wherein the wearable device further comprises a speaker and an actuator,
wherein the instructions, when executed by the at least one processor individually or collectively, cause the wearable device to:
when executing a software application for monitoring a body posture, display, through the display, a screen to set a manner of a notification, and
based on receiving a user input with respect to the screen, when identifying that the time is greater than the threshold time, perform at least one among displaying the first UI object through the display, outputting an audio signal corresponding to the notification through the speaker, and controlling the actuator for providing a vibration notification.

10. The wearable device of claim 1, wherein the instructions, when executed by the at least one processor individually or collectively, cause the wearable device to:
when executing a software application for monitoring a body posture, display, through the display, a screen to set the threshold time, and
based on receiving a user input with respect to the screen, determine the threshold time.

11. The wearable device of claim 1, wherein the wearable device further comprises an actuator,
wherein the instructions, when executed by the at least one processor individually or collectively, cause the wearable device to:
based on obtaining the distance outside the range:
identify the time during which the distance has been maintained outside the range,
based on identifying the time greater than the threshold time, control the actuator to provide a vibration notification at a first intensity, and
based on identifying another time greater than the time, control the actuator to provide the vibration notification at a second intensity.

12. The wearable device of claim 1, wherein the range is set based on a first value for a first ratio of the reference distance and a second value for a second ratio of the reference distance.

13. The wearable device of claim 1, wherein the information is obtained based on a received signal strength indicator (RSSI) using the communication circuitry.

14. A non-transitory computer readable storage medium storing one or more programs, the one or more programs comprising instructions to, when executed by a wearable device with a display and communication circuitry, cause the wearable device to:
while being connected to an external electronic device through the communication circuitry, obtain information associated with a distance between the wearable device and the external electronic device,
by using the information, identify whether the distance is included in a range that is set according to a reference distance between the wearable device and the external electronic device,
based on obtaining the distance outside the range:
identify a time during which the distance has been maintained outside the range, and
based on identifying the time greater than a threshold time, display, through the display, a first user interface (UI) object indicating that a posture of a user of the wearable device is corresponding to a first posture,
wherein the reference distance is obtained before the information is obtained.

15. The non-transitory computer readable storage medium of claim 14, wherein the range is set to define the first posture of the user wearing the wearable device and the external electronic device,
wherein the one or more programs comprise instructions to, when executed by the wearable device, cause the wearable device to:
based on identifying that the distance is included in the range:
identify another time during which the distance has been maintained in the range, and
based on identifying the another time greater than another threshold time, display, through the display, a second UI object indicating that the posture of the user corresponds to the second posture.

16. The non-transitory computer readable storage medium of claim 14, wherein the one or more programs comprise instructions to, when executed by the wearable device, cause the wearable device to:
by using the information, identify acceleration of the external electronic device at least based on an acceleration sensor included in the external electronic device, the acceleration sensor being periodically enabled according to a first period;
based on identifying the acceleration lower than a threshold acceleration, transmit, through the communication circuitry to the external electronic device, a signal instructing to change a period of the acceleration sensor to a second period longer than the first period, and
based on identifying the acceleration higher than the threshold acceleration, transmit, through the communication circuitry to the external electronic device, another signal instructing to maintain the period of the acceleration sensor as the first period.

17. The non-transitory computer readable storage medium of claim 14, wherein the wearable device further comprises an acceleration sensor,
wherein the one or more programs comprise instructions to, when executed by the wearable device, cause the wearable device to:
identify, through the acceleration sensor, acceleration of the wearable device,
based on a determination that the acceleration is higher than a threshold acceleration, obtain, in a first period, the information, and
based on a determination that the acceleration is lower than the threshold acceleration, obtain, in a second period longer than the first period, the information.

18. The non-transitory computer readable storage medium of claim 14, wherein the wearable device further comprises a rechargeable battery,
wherein the one or more programs comprise instructions to, when executed by the wearable device, cause the wearable device to:
based on a determination that a state of charge (SoC) of the rechargeable battery is higher than a threshold SoC, obtain, in a first period, the information, and
based on a determination that the SoC of the rechargeable battery is lower than the threshold SoC, obtain, in a second period longer than the first period, the information.

19. The non-transitory computer readable storage medium of claim 14, wherein the one or more programs comprise instructions to, when executed by the wearable device, cause the wearable device to:
while displaying the first UI object, obtain other information associated with the distance between the wearable device and the external electronic device; and
based on identifying another distance indicated by the other information being included in the range, display, through the display, a third UI object indicating that the posture of the user corresponds to the second posture.

20. A method executed in a wearable device including a display and communication circuitry, the method comprising:
while being connected to an external electronic device through the communication circuitry, obtaining information associated with a distance between the wearable device and the external electronic device,
by using the information, identifying whether the distance is included in a range that is set according to a reference distance between the wearable device and the external electronic device,
based on obtaining the distance outside the range:
identifying a time during which the distance has been maintained outside the range, and
based on identifying the time greater than a threshold time, displaying, through the display, a first user interface (UI) object indicating that a posture of a user of the wearable device is corresponding to a first posture, and
wherein the reference distance is obtained before the information is obtained.
